SEQUENCE LISTING (57 sequences)

SEQ ID NO: 1 - RNA, 41 nt, synthetic construct
  source 1..41: mol_type = other RNA
taatttctactaagtgtagatggatttgtgttcacgctcac

SEQ ID NO: 2 - DNA, 23 bp, synthetic construct
  source 1..23: mol_type = other DNA
ccaatcctgtcacctctgactaa

SEQ ID NO: 3 - DNA, 22 bp, synthetic construct
  source 1..22: mol_type = other DNA
ccaatcctgtcacctctgacca

SEQ ID NO: 4 - DNA, 23 bp, synthetic construct
  source 1..23: mol_type = other DNA
ccaatcctgtcacctttgactaa

SEQ ID NO: 5 - DNA, 17 bp, synthetic construct
  source 1..17: mol_type = other DNA
gcgtctacgctgcagtc

SEQ ID NO: 6 - DNA, 20 bp, synthetic construct
  source 1..20: mol_type = other DNA
  modified_base 1: mod_base = OTHER; note = 5cy55
  modified_base 20: mod_base = OTHER; note = 3BHQ_2
ctcactgggcacggtgagcg

SEQ ID NO: 7 - DNA, 20 bp, synthetic construct
  source 1..20: mol_type = other DNA
  modified_base 1: mod_base = OTHER; note = 5TYE705
  modified_base 20: mod_base = OTHER; note = 3BHQ_2
ctcactgggcacggtgagcg

SEQ ID NO: 8 - DNA, 87 bp, Influenza A
  source 1..87: mol_type = genomic DNA
ccaatcttgtcacctctgactaagggaattttaggatttgtgttcacgctcaccgtgcccagtgagcgaggactgcagcgtagacgc

SEQ ID NO: 9 - DNA, 87 bp, Influenza A; identical to SEQ ID NO: 8
  source 1..87: mol_type = genomic DNA

SEQ ID NO: 10 - DNA, 87 bp, synthetic construct
  source 1..87: mol_type = other DNA
ccaatcttgtcacctctgactaagggaatcttaggatttgtgttcacgctcaccgtgcccagtgagcgaggactgcagcgtagacgc

SEQ ID NO: 11 - DNA, 87 bp, synthetic construct
  source 1..87: mol_type = other DNA
ccaatcttgtcacctctgactaagggaattctaggatttgtgttcacgctcaccgtgcccagtgagcgaggactgcagcgtagacgc

SEQ ID NO: 12 - DNA, 87 bp, synthetic construct
  source 1..87: mol_type = other DNA
ccaatcttgtcacctctgactaagggaatttaaggatttgtgttcacgctcaccgtgcccagtgagcgaggactgcagcgtagacgc

SEQ ID NO: 13 - DNA, 87 bp, synthetic construct
  source 1..87: mol_type = other DNA
ccaatcttgtcacctctgactaagggaatttttggatttgtgttcacgctcaccgtgcccagtgagcgaggactgcagcgtagacgc

SEQ ID NO: 14 - DNA, 87 bp, synthetic construct
  source 1..87: mol_type = other DNA
ccaatcttgtcacctctgactaagggaattttacgatttgtgttcacgctcaccgtgcccagtgagcgaggactgcagcgtagacgc

SEQ ID NO: 15 - DNA, 87 bp, synthetic construct
  source 1..87: mol_type = other DNA
ccaatcttgtcacctctgactaagggaattttagtatttgtgttcacgctcaccgtgcccagtgagcgaggactgcagcgtagacgc

SEQ ID NO: 16 - DNA, 87 bp, synthetic construct
  source 1..87: mol_type = other DNA
ccaatcttgtcacctctgactaagggaattttaggctttgtgttcacgctcaccgtgcccagtgagcgaggactgcagcgtagacgc

SEQ ID NO: 17 - DNA, 87 bp, synthetic construct
  source 1..87: mol_type = other DNA
ccaatcttgtcacctctgactaagggaattttaggaattgtgttcacgctcaccgtgcccagtgagcgaggactgcagcgtagacgc

SEQ ID NO: 18 - DNA, 87 bp, synthetic construct
  source 1..87: mol_type = other DNA
ccaatcttgtcacctctgactaagggaattttaggatgtgtgttcacgctcaccgtgcccagtgagcgaggactgcagcgtagacgc

SEQ ID NO: 19 - DNA, 87 bp, synthetic construct
  source 1..87: mol_type = other DNA
ccaatcttgtcacctctgactaagggaattttaggattcgtgttcacgctcaccgtgcccagtgagcgaggactgcagcgtagacgc

SEQ ID NO: 20 - DNA, 87 bp, synthetic construct
  source 1..87: mol_type = other DNA
ccaatcttgtcacctctgactaagggaattttaggatttttgttcacgctcaccgtgcccagtgagcgaggactgcagcgtagacgc

SEQ ID NO: 21 - DNA, 87 bp, synthetic construct
  source 1..87: mol_type = other DNA
ccaatcttgtcacctctgactaagggaattttaggatttgagttcacgctcaccgtgcccagtgagcgaggactgcagcgtagacgc

SEQ ID NO: 22 - DNA, 87 bp, synthetic construct
  source 1..87: mol_type = other DNA
ccaatcttgtcacctctgactaagggaattttaggatttgtattcacgctcaccgtgcccagtgagcgaggactgcagcgtagacgc

SEQ ID NO: 23 - DNA, 87 bp, synthetic construct
  source 1..87: mol_type = other DNA
ccaatcttgtcacctctgactaagggaattttaggatttgtgatcacgctcaccgtgcccagtgagcgaggactgcagcgtagacgc

SEQ ID NO: 24 - DNA, 87 bp, synthetic construct
  source 1..87: mol_type = other DNA
ccaatcttgtcacctctgactaagggaattttaggatttgtgtccacgctcaccgtgcccagtgagcgaggactgcagcgtagacgc

SEQ ID NO: 25 - DNA, 87 bp, synthetic construct
  source 1..87: mol_type = other DNA
ccaatcttgtcacctctgactaagggaattttaggatttgtgtttacgctcaccgtgcccagtgagcgaggactgcagcgtagacgc

SEQ ID NO: 26 - DNA, 87 bp, synthetic construct
  source 1..87: mol_type = other DNA
ccaatcttgtcacctctgactaagggaattttaggatttgtgttcccgctcaccgtgcccagtgagcgaggactgcagcgtagacgc

SEQ ID NO: 27 - DNA, 87 bp, synthetic construct
  source 1..87: mol_type = other DNA
ccaatcttgtcacctctgactaagggaattttaggatttgtgttcaggctcaccgtgcccagtgagcgaggactgcagcgtagacgc

SEQ ID NO: 28 - DNA, 87 bp, synthetic construct
  source 1..87: mol_type = other DNA
ccaatcttgtcacctctgactaagggaattttaggatttgtgttcactctcaccgtgcccagtgagcgaggactgcagcgtagacgc

SEQ ID NO: 29 - DNA, 87 bp, synthetic construct
  source 1..87: mol_type = other DNA
ccaatcttgtcacctctgactaagggaattttaggatttgtgttcacgatcaccgtgcccagtgagcgaggactgcagcgtagacgc

SEQ ID NO: 30 - DNA, 87 bp, synthetic construct
  source 1..87: mol_type = other DNA
ccaatcttgtcacctctgactaagggaattttaggatttgtgttcacgcccaccgtgcccagtgagcgaggactgcagcgtagacgc

SEQ ID NO: 31 - DNA, 87 bp, synthetic construct
  source 1..87: mol_type = other DNA
ccaatcttgtcacctctgactaagggaattttaggatttgtgttcacgcttaccgtgcccagtgagcgaggactgcagcgtagacgc

SEQ ID NO: 32 - DNA, 87 bp, synthetic construct
  source 1..87: mol_type = other DNA
ccaatcttgtcacctctgactaagggaattttaggatttgtgttcacgctcgccgtgcccagtgagcgaggactgcagcgtagacgc

SEQ ID NO: 33 - DNA, 87 bp, synthetic construct
  source 1..87: mol_type = other DNA
ccaatcttgtcacctctgactaagggaattttaggatttgtgttcacgctcatcgtgcccagtgagcgaggactgcagcgtagacgc

SEQ ID NO: 34 - DNA, 87 bp, synthetic construct
  source 1..87: mol_type = other DNA
ccaatcttgtcacctctgactaagggaattttaggatttgtgttcacgctcacagtgcccagtgagcgaggactgcagcgtagacgc

SEQ ID NO: 35 - DNA, 87 bp, synthetic construct
  source 1..87: mol_type = other DNA
ccaatcttgtcacctctgactaagggaattttaggatttgtgttcacgctcaccttgcccagtgagcgaggactgcagcgtagacgc

SEQ ID NO: 36 - DNA, 87 bp, synthetic construct
  source 1..87: mol_type = other DNA
ccaatcttgtcacctctgactaagggaattttaggatttgtgttcacgctcaccgggcccagtgagcgaggactgcagcgtagacgc

SEQ ID NO: 37 - DNA, 87 bp, synthetic construct
  source 1..87: mol_type = other DNA
ccaatcttgtcacctctgactaagggaattttaggatttgtgttcacgctcaccgtccccagtgagcgaggactgcagcgtagacgc

SEQ ID NO: 38 - DNA, 87 bp, synthetic construct
  source 1..87: mol_type = other DNA
ccaatcttgtcacctctgactaagggaattttaggatttgtgttcacgctcaccgtgaccagtgagcgaggactgcagcgtagacgc

SEQ ID NO: 39 - DNA, 87 bp, synthetic construct
  source 1..87: mol_type = other DNA
ccaatcttgtcacctctgactaagggaattttaggatttgtgttcacgctcaccgtgcgcagtgagcgaggactgcagcgtagacgc

SEQ ID NO: 40 - DNA, 87 bp, synthetic construct
  source 1..87: mol_type = other DNA
ccaatcttgtcacctctgactaagggaattttaggatttgtgttcacgctcaccgtgccgagtgagcgaggactgcagcgtagacgc

SEQ ID NO: 41 - DNA, 87 bp, synthetic construct
  source 1..87: mol_type = other DNA
ccaatcttgtcacctctgactaagggaattttaggatttgtgttcacgctcaccgtgccctgtgagcgaggactgcagcgtagacgc

SEQ ID NO: 42 - DNA, 87 bp, synthetic construct
  source 1..87: mol_type = other DNA
ccaatcttgtcacctctgactaagggaattttaggatttgtgttcacgctcaccgtgcccactgagcgaggactgcagcgtagacgc

SEQ ID NO: 43 - DNA, 87 bp, synthetic construct
  source 1..87: mol_type = other DNA
ccaatcttgtcacctctgactaagggaattttaggatttgtgttcacgctcaccgtgcccagcgagcgaggactgcagcgtagacgc

SEQ ID NO: 44 - DNA, 87 bp, synthetic construct
  source 1..87: mol_type = other DNA
ccaatcttgtcacctctgactaagggaattttaggatttgtgttcacgctcaccgtgcccagtcagcgaggactgcagcgtagacgc

SEQ ID NO: 45 - DNA, 87 bp, synthetic construct
  source 1..87: mol_type = other DNA
ccaatcttgtcacctctgactaagggaattttaggatttgtgttcacgctcaccgtgcccagtgtgcgaggactgcagcgtagacgc

SEQ ID NO: 46 - DNA, 87 bp, synthetic construct
  source 1..87: mol_type = other DNA
ccaatcttgtcacctctgactaagggaattttaggatttgtgttcacgctcaccgtgcccagtgaccgaggactgcagcgtagacgc

SEQ ID NO: 47 - DNA, 21 bp, synthetic construct
  source 1..21: mol_type = other DNA
cgagcgtcttaatgaaggaca

SEQ ID NO: 48 - DNA, 22 bp, synthetic construct
  source 1..22: mol_type = other DNA
tgctcttgaccaaattgggata

SEQ ID NO: 49 - DNA, 27 bp, synthetic construct
  source 1..27: mol_type = other DNA
  modified_base 1: mod_base = OTHER; note = 5TEX615
  modified_base 27: mod_base = OTHER; note = 3BHQ_2
aagccaattcgagcagctgaaactgcg

SEQ ID NO: 50 - RNA, 64 nt, synthetic construct
  source 1..64: mol_type = other RNA
gatttagactaccccaaaaacgaaggggactaaaacaggacattcaaagccaattcgagcagct

SEQ ID NO: 51 - RNA, 64 nt, synthetic construct
  source 1..64: mol_type = other RNA
gatttagactaccccaaaaacgaaggggactaaaacaaagccaattcgagcagctgaaactgcg

SEQ ID NO: 52 - RNA, 64 nt, synthetic construct
  source 1..64: mol_type = other RNA
gatttagactaccccaaaaacgaaggggactaaaaccgagcagctgaaactgcggtgggagtct

SEQ ID NO: 53 - RNA, 64 nt, synthetic construct
  source 1..64: mol_type = other RNA
gatttagactaccccaaaaacgaaggggactaaaaccttgaccaaattgggataagactcccac

SEQ ID NO: 54 - RNA, 59 nt, synthetic construct
  source 1..59: mol_type = other RNA
gaccaccccaaaaatgaaggggactaaaacaaggacattcaaagccaattcgagcagct

SEQ ID NO: 55 - RNA, 59 nt, synthetic construct
  source 1..59: mol_type = other RNA
gaccaccccaaaaatgaaggggactaaaacaaaagccaattcgagcagctgaaactgcg

SEQ ID NO: 56 - RNA, 59 nt, synthetic construct
  source 1..59: mol_type = other RNA
gaccaccccaaaaatgaaggggactaaaacacgagcagctgaaactgcggtgggagtct

SEQ ID NO: 57 - RNA, 59 nt, synthetic construct
  source 1..59: mol_type = other RNA
gaccaccccaaaaatgaaggggactaaaacacttgaccaaattgggataagactcccac

(19) United States
(12) Patent Application Publication
(10) Pub. No.: US 2026/0159881 A1
(43) Pub. Date: Jun. 11, 2026

(54) DEVICES AND APPLICATIONS OF COUPLED NUCLEIC ACID AMPLIFICATION TESTS AND CRISPR SYSTEMS

(71) Applicant: Roche Molecular Systems, Inc., Pleasanton, CA (US)

(72) Inventors: He Wang, Pleasanton, CA (US); Jinshuang Lu, Pleasanton, CA (US); Yu Tian, Pleasanton, CA (US); Xiang Li, Pleasanton, CA (US); Renjian Huang, Pleasanton, CA (US); Beiyang Ma, Pleasanton, CA (US); Fangnian Wang, Pleasanton, CA (US)

(21) Appl. No.: 19/539,370

(22) Filed: Feb. 13, 2026

Related U.S. Application Data
(63) Continuation of application No. PCT/EP2024/072005, filed on Aug. 2, 2024.
(60) Provisional application No. 63/532,651, filed on Aug. 14, 2023.

(51) Int. Cl. C12Q 1/686; C12Q 1/34; C12Q 1/48; C12Q 1/6848; C12Q 1/6888; C12Q 1/70

(52) U.S. Cl. CPC C12Q 1/686; C12Q 1/34; C12Q 1/48; C12Q 1/6848; C12Q 1/6888; C12Q 1/701; C12Y 207/07007; C12Y 207/07049; C12Y 301/00; C12Y 302/02027; C12Q 2600/16

(57) ABSTRACT

The present disclosure relates to devices and applications of nucleic acid amplification tests (NAAT) and CRISPR systems integrated into one system.

### RELATED APPLICATIONS

This application is a continuation of International Patent Application No. PCT/EP2024/072005, filed Aug. 2, 2024, which claims priority to U.S. Provisional Patent Application No. 63/532,651, filed Aug. 14, 2023, both of which are hereby incorporated herein by reference.

### FIELD OF INVENTION

This disclosure relates generally to devices, systems, and methods for coupling nucleic acid amplification tests (NAAT) and CRISPR systems integrated into one system to improve targeted detection of microorganisms and/or nucleic acids in a sample.

### BACKGROUND

The swift and precise diagnosis of a disease is crucial for effective treatment and preventing long-term complications. Nucleic acid-based biomarkers associated with diseases play a pivotal role in diagnostics as DNA and RNA can be amplified from minuscule quantities, facilitating their highly specific detection through complementary nucleotide pairing. Indeed, nucleic acid-based diagnostics have emerged as the gold standard for various acute and chronic conditions, particularly those caused by infectious diseases.

Quantitative polymerase chain reaction (qPCR) and sequencing-based nucleic acid diagnostics have gained widespread acceptance and are frequently employed in clinical laboratories. Nevertheless, traditional PCR techniques require trained personnel for preparing the samples and setting up the amplification reactions and thermocyclers for shuffling back-and-fourth between various temperatures. Isothermal nucleic acid amplification strategies eliminate the need for thermal cyclers, on the other hand it may lead to lower detection specificity due to non-specific amplification. Certain additional readouts, incorporated with some fluorescent probes, oligo strand-displacement probes, or molecular beacons, may enhance specificity, but still relies on amplification of a template.

Clustered regularly interspaced short palindromic repeats (CRISPR)-based diagnostics could address some of these limitations. While various CRISPR-Cas systems exist within different species of archaea and bacteria, they share a common reliance on a crRNA to direct CRISPR proteins in identifying and cutting specific nucleic acid targets. The crRNA can be customized to target a particular DNA or RNA region by binding to a complementary sequence, which, in certain systems, is restricted to regions near a protospacer adjacent motif (PAM) or protospacer flanking sequence.

There is an unprecedented need for systems and methods that can improve our ability to accurately identify the microorganism causing an illness in a practical and timely manner. The present disclosure addresses this need by devising dual-detection systems that combine distinct technologies to detect a microorganism, thus providing an output that greatly improves performance of the existing technologies.

### SEQUENCE LISTING

This application contains a sequence listing provided in Table 1. The Sequence Listing associated with this application is further provided in xml format. The name of the text file containing the Sequence Listing is P38755_WO_Sequence_Listing.xml. The xml file is 57.143 bytes, and was created on Jul. 24, 2024, the XML file is hereby incorporated by reference.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In some aspects, provided here are devices, systems, and methods for coupling nucleic acid amplification tests and CRISPR systems thus improving targeted detection of microorganisms in a sample.

In some aspects, the disclosure provides a device comprising: an assay tube subdivided into a plurality of subsections, whereby a first subsection is connected to an openable cap, whereby an opening accessible from the openable cap in the first subsection is adapted for receiving a sample, whereby the first subsection is sealed from a subsequent subsection along the tube; a second subsection comprising a solution with an internal control, whereby the second subsection is sealed from a subsequent subsection along the tube; a third subsection comprising a solution with magnetic beads in solution, whereby the third subsection is sealed from a subsequent subsection along the tube; a fourth subsection comprising a solution with a lysis buffer, whereby the fourth subsection is sealed from a subsequent subsection along the tube; a fifth subsection comprising a solution with a wash buffer, whereby the fifth subsection is sealed from a subsequent subsection along the tube; a sixth subsection comprising a solution with an elution buffer, whereby the sixth subsection is sealed from a subsequent subsection along the tube; a seventh subsection comprising a solution with a first PCR master mix, hereby the seventh subsection is sealed from a subsequent subsection along the tube; an eighth subsection comprising a solution with a second PCR master mix, whereby the eighth subsection is sealed from a subsequent subsection along the tube; and a ninth tube comprising a solution with a CRISPR enzyme, whereby the ninth subsection is sealed from an end of the tube. The assay tube typically has a length of 106±21 millimeters and is subdivided into sections for receiving suitable volume of reagents for one or more stages of the NAAT and/or CRISPR reactions. In some configurations, the first subsection is adapted for receiving a volume of about 200±40 microliters of sample; the second subsection comprises 12.5±2.5 microliters of the solution with the internal control reagents; the third subsection comprises 12.5±2.5 microliters of the solution with the magnetic beads; the fourth subsection comprises 215±43 microliters of the solution with the lysis buffer; the fifth subsection comprises 240±48 microliters of the solution with the wash buffer; the sixth subsection comprises 50±10 microliters of the solution with the elution buffer; the seventh subsection comprises 30±6 microliters of the solution with the first PCR master mix; the eight subsection comprises 15±3 microliters of the solution with the second PCR master mix; and the ninth subsection comprises 60±12 microliters of the solution with the CRISPR enzyme. This disclosure contemplates scenarios where certain configurations of the device could include duplicate sections and scenarios where certain buffer reagents could be combined. In certain configurations, the internal control reagents are control materials for reverse transcription-polymerase chain reaction (RT-PCR) reagents. In certain configurations, the magnetic bead reagents are Liat Magnetic Particles (Roche®), used for sample preparation and nucleic acid extraction. In certain configurations, the lysis buffer is Liat® Generic Lysis Buffer 1. In certain configurations the wash buffer is Cobas® Omni Wash Buffer. In certain configurations the elution buffer is FRTA Elution Buffer (Roche®). In certain configurations the device comprises two or more PCR master mixes. The first PCR master mix can be a solution comprising a reaction buffer, dNTP, RT-PCR primers, MMLV RT polymerase, Uracil-DNA glycosylase. The second PCR master mix can be a solution comprising a reaction buffer, Z05 polymerase and its aptamer, RT-PCR primers. In certain configurations the assay tube further comprises a tenth subsection comprising a solution with a CRISPR enzyme, whereby the tenth subsection is sealed from an end of the assay tube. The CRISPR enzyme can be part of a CRISPR system adapted for specifically cleaving a plurality of nucleic acids that are amplified by PCR reaction. In certain configurations, the CRISPR enzyme is a CRISPR-Cas enzyme with trans-cleavage activity such as Cas12 and Cas13. A CRISPR master mix described herein comprises a guide RNA for targeting a target sequence of a microorganism, substrates, and metal ion co-factors. In some embodiments, the assay tube is mounted on a frame, e.g., an open frame. In certain configurations either a left side or a right side of the frame can comprises a grading that is, e.g., adapted for facilitating a sliding of the device into a system that provides a signal readout. In certain configurations the sample preparation reagents packed within the device extract nucleic acid materials from samples such as influenza A virus, influenza B virus, Respiratory Syncytial Virus (RSV), and SARS-CoV-2 virus. The assay tube can be a thermoplastic tube, e.g., a tube that is made from polypropylene. In some aspects, the disclosure provides a method for detecting a target nucleic acid sequence with an assay tube described herein.

In some aspects, the disclosure provides a method for dual-detection of a target microorganism comprising: (a) amplifying a first target nucleic acid sequence in a nucleic acid amplification reaction and detecting a signal from the nucleic acid amplification of the first target nucleic acid sequence; (b) detecting a second target nucleic acid sequence with a CRISPR system targeting the second target nucleic acid sequence and detecting a signal from the second target nucleic acid activation and substrate cleavage by the CRISPR system; (c) comparing the signal detected from the nucleic acid amplification of the first target nucleic acid sequence with an amplification threshold and comparing the signal detected from the second target nucleic acid activation and substrate cleavage by the CRISPR system with a CRISPR threshold; wherein the first target nucleic acid sequence and the second target nucleic acid sequence are sequences from the target microorganism; whereby the target nucleic acid sequence is dually-detected when the signal detected from the first target nucleic acid amplification reaction and the signal detected from the second target nucleic acid activation and substrate cleavage by the CRISPR system are both above the threshold. The present disclosure contemplates embodiments wherein the nucleic acid amplification reaction is a polymerase chain reaction (PCR); a loop mediated isothermal amplification (LAMP) reaction; a recombinase polymerase amplification (RPA) reaction; a nucleic acid sequence based amplification (NASBS) reaction; a transcription mediated amplification (TMA) reaction; a rolling circle amplification (RCA) reaction; a strand displacement amplification (SDA) reaction; a nicking and extension amplification reaction (NEAR); an exponential amplification reaction (EXPAR); a multiple displacement amplification (MDA) reaction; a helicase dependent amplification (HAD) reaction; or a hybridization chain reaction (HCR). The present disclosure contemplates embodiments wherein the CRISPR system is a system with trans-cleavage nuclease activity, such as a Cas9 system, a Cas12 system, or a Cas13 system. In some embodiments, the first and the second target nucleic acid sequences are FluA. In some embodiments, a region of the first target nucleic acid amplification sequence is amplified during the nucleic acid amplification reaction; thereby producing an amplicon that comprises the second target nucleic acid sequence. In some instances, the first target nucleic acid sequence comprises a plurality of regions comprising the second target nucleic acid sequence(s). In some instances, activation and substrate cleavage of the plurality of second target nucleic acid sequence(s) further amplifies the signal detected from the second target nucleic acid by a factor that is directly proportional to the number of the plurality of second target nucleic acid sequences. In some instances, the method comprises amplifying a plurality of first target nucleic acid sequences for multiplex detection of a plurality of microorganisms, e.g., if first nucleic acid amplification is 3-plex (3 channels), with CRISPR it will become 6-plex (3 original channels+3 additional). In some instances, the method comprises amplifying a plurality of first target nucleic acid sequences for multiplex detection of a same microorganisms, thereby increasing sensitivity by amplifying different regions of the same target. In some instances, the amplification threshold provides a sensitivity of at least 120 copies/mL. In some instances, the CRISPR threshold provides a sensitivity of at least 120 copies/mL (same sensitivity but with improved signal strength). In some instances, the method further comprises a step of cleaving a plurality of nucleic acids with a non-specific nuclease after detection of the amplification threshold and the CRISPR threshold thereby reducing nucleic acid contamination in the system.

Embodiment 1: A device comprising:
an assay tube subdivided into a plurality of subsections, whereby:
a first subsection is connected to an openable cap, whereby an opening accessible from the openable cap in the first subsection is adapted for receiving a sample, whereby the first subsection is sealed from a subsequent subsection along the assay tube;
a second subsection comprising a solution with an internal control, whereby the second subsection is sealed from a subsequent subsection along the assay tube;
a third subsection comprising a solution with magnetic beads in solution, whereby the third subsection is sealed from a subsequent subsection along the assay tube;
a fourth subsection comprising a solution with a lysis buffer, whereby the fourth subsection is sealed from a subsequent subsection along the assay tube;
a fifth subsection comprising a solution with a wash buffer, whereby the fifth subsection is sealed from a subsequent subsection along the assay tube;
a sixth subsection comprising a solution with an elution buffer, whereby the sixth subsection is sealed from a subsequent subsection along the assay tube;
a seventh subsection comprising a solution with a first PCR master mix, hereby the seventh subsection is sealed from a subsequent subsection along the assay tube;
an eighth subsection comprising a solution with a second PCR master mix, whereby the eighth subsection is sealed from a subsequent subsection along the assay tube; and
a ninth tube comprising a solution with a CRISPR enzyme, whereby the ninth subsection is sealed from an end of the assay tube.

Embodiment 2: The device of embodiment 1, wherein the tube has a length of 106±21 millimeters.

Embodiment 3: The device of embodiments 1 or 2, wherein the first subsection is adapted for receiving a volume of 200±40 microliters.

Embodiment 4: The device of any of embodiments 1 to 3, wherein the second subsection comprises 12.5±2.5 microliters of the solution with the internal control reagents.

Embodiment 5: The device of any of embodiments 1 to 4, wherein the third subsection comprises 12.5±2.5 microliters of the solution with the magnetic beads.

Embodiment 6: The device of any of embodiments 1 to 5, wherein the fourth subsection comprises 215±43 microliters of the solution with the lysis buffer.

Embodiment 7: The device of any of embodiments 1 to 6, wherein the fifth subsection comprises 240±48 microliters of the solution with the wash buffer.

Embodiment 8: The device of any of embodiments 1 to 7, wherein the sixth subsection comprises 50±10 microliters of the solution with the elution buffer.

Embodiment 9: The device of any of embodiments 1 to 8, wherein the seventh subsection comprises 30±6 microliters of the solution with the first PCR master mix.

Embodiment 10: The device of any of embodiments 1 to 9, wherein the eight subsection comprises 15±3 microliters of the solution with the second PCR master mix.

Embodiment 11: The device of any of embodiments 1 to 10, wherein the ninth subsection comprises 60±12 microliters of the solution with the CRISPR enzyme.

Embodiment 12: The device of any of embodiments 1 to 11, wherein the internal control reagents are control materials for the reverse transcription-polymerase chain reaction (RT-PCR) reagents.

Embodiment 13: The device of any of embodiments 1 to 12, wherein the magnetic beads are Liat Magnetic Particles.

Embodiment 14: The device of any of embodiments 1 to 13, wherein the lysis buffer is a Liat Lysis Buffer.

Embodiment 15: The device of any of embodiments 1 to 14, wherein the wash buffer is a cobas Omni Wash Buffer.

Embodiment 16: The device of any of embodiments 1 to 15, wherein the elution buffer is an FRTA Elution Buffer.

Embodiment 17: The device of any of embodiments 1 to 16, wherein the first PCR master mix is a solution consisting of reaction buffer, dNTP, RT-PCR primers, MMLV RT polymerase, Uracil-DNA glycosylase.

Embodiment 18: The device of any of embodiments 1 to 17, wherein the second PCR master mix is a solution consisting of reaction buffer, Z05 polymerase and its aptamer, RT-PCR primers.

Embodiment 19: The device of any of embodiments 1 to 18, wherein the CRISPR enzyme is a solution consisting of CRISPR-Cas enzymes with trans-cleavage activity such as Cas12 and Cas13, guide RNA, substrates, metal ion co-factors.

Embodiment 20: The device of any of embodiments 1 to 19, wherein the assay tube is mounted on a frame.

Embodiment 21: The device of embodiment 20, wherein the frame is an open frame.

Embodiment 22: The device of embodiment 21, wherein a right side of the frame comprises a grading.

Embodiment 23: The device of embodiment 22, wherein the grading is adapted for facilitating a sliding of the device into a system that provides a signal readout.

Embodiment 24: The device of any of embodiments 1 to 23, wherein the sample preparation reagents extracts nucleic acid materials from influenza A virus, influenza B virus, Respiratory Syncytial Virus (RSV), and SARS-CoV-2 virus.

Embodiment 25: The device of any of embodiments 1 to 24, wherein the assay tube is a thermoplastic tube.

Embodiment 26: The device of embodiment 25, wherein the thermoplastic tube is made from polypropylene.

Embodiment 27: The device of any of embodiments 1 to 26, wherein the tube further comprises a tenth subsection comprising a solution with a CRISPR enzyme, whereby the tenth subsection is sealed from an end of the tube.

Embodiment 28: The device of embodiment 27, wherein the CRISPR enzyme is part of a CRISPR system adapted for specifically cleaving a plurality of nucleic acids that are amplified by PCR reaction.

Embodiment 29: A method for dual-detection of a target microorganism comprising:
(a) amplifying a first target nucleic acid sequence in a nucleic acid amplification reaction and detecting a signal from the nucleic acid amplification of the first target nucleic acid sequence;
(b) detecting a second target nucleic acid sequence with a CRISPR system targeting the second target nucleic acid sequence and detecting a signal from the second target nucleic acid activation and substrate cleavage by the CRISPR system;
(c) comparing the signal detected from the nucleic acid amplification of the first target nucleic acid sequence with an amplification threshold and comparing the signal detected from the second target nucleic acid activation and substrate cleavage by the CRISPR system with a CRISPR threshold;
wherein the first target nucleic acid sequence and the second target nucleic acid sequence are sequences from the target microorganism;
whereby the target nucleic acid sequence is dually-detected when the signal detected from the first target nucleic acid amplification reaction and the signal detected from the second target nucleic acid activation and substrate cleavage by the CRISPR system are both above the threshold.

Embodiment 30: The method of embodiment 29, wherein the nucleic acid amplification reaction is a polymerase chain reaction (PCR).

Embodiment 31: The method of embodiment 29, wherein the nucleic acid amplification reaction is a loop mediated isothermal amplification (LAMP) reaction.

Embodiment 32: The method of embodiment 29, wherein the nucleic acid amplification reaction is a recombinase polymerase amplification (RPA) reaction.

Embodiment 33: The method of embodiment 29, wherein the nucleic acid amplification reaction is a nucleic acid sequence based amplification (NASBS) reaction.

Embodiment 34: The method of embodiment 29, wherein the nucleic acid amplification reaction is a transcription mediated amplification (TMA) reaction.

Embodiment 35: The method of embodiment 29, wherein the nucleic acid amplification reaction is a rolling circle amplification (RCA) reaction.

Embodiment 36: The method of embodiment 29, wherein the nucleic acid amplification reaction is a strand displacement amplification (SDA) reaction.

Embodiment 37: The method of embodiment 29, wherein the nucleic acid amplification reaction is a nicking and extension amplification reaction (NEAR).

Embodiment 38: The method of embodiment 29, wherein the nucleic acid amplification reaction is an exponential amplification reaction (EXPAR).

Embodiment 39: The method of embodiment 29, wherein the nucleic acid amplification reaction is a multiple displacement amplification (MDA) reaction.

Embodiment 40: The method of embodiment 29, wherein the nucleic acid amplification reaction is a helicase dependent amplification (HAD) reaction.

Embodiment 41: The method of embodiment 29, wherein the nucleic acid amplification reaction is a hybridization chain reaction (HCR).

Embodiment 42: The method of any of embodiments 29 to 41, wherein the CRISPR system is a system with trans-cleavage nuclease activity.

Embodiment 43: The method of embodiment 42, wherein the system with trans-cleavage activity is a Cas12 system.

Embodiment 44: The method of embodiment 42, wherein the system with trans-cleavage activity is a Cas13 system.

Embodiment 45: The method of any of embodiments 29 to 44, wherein the first target nucleic acid sequence is a FluA sequence.

Embodiment 46: The method of any of embodiments 29 to 44, wherein the second target nucleic acid sequence is FluA sequence.

Embodiment 47: The method of any of embodiments 29 to 46, wherein a region of the first target nucleic acid amplification sequence comprises the second target nucleic acid sequence.

Embodiment 48: The method of embodiment 47, wherein the first target nucleic acid sequence comprises a plurality of regions comprising the second target nucleic acid sequence(s).

Embodiment 49: The method of embodiment 48, wherein activation and substrate cleavage of the plurality of second target nucleic acid sequence(s) further amplifies the signal detected from the second target nucleic acid by a factor that is directly proportional to the number of the plurality of second target nucleic acid sequences.

Embodiment 50: The method of any of embodiments 29 to 49, wherein the method comprises amplifying a plurality of first target nucleic acid sequences for multiplex detection of a plurality of microorganisms.

Embodiment 51: The method of any of embodiments 29 to 49, wherein the method comprises amplifying a plurality of first target nucleic acid sequences for multiplex detection of the same microorganisms.

Embodiment 52: The method of any of embodiments 29 to 51, wherein the amplification threshold provides a sensitivity of at least 120 copies/mL.

Embodiment 53: The method of any of embodiments 29 to 52, wherein the CRISPR threshold provides a sensitivity of at least 120 copies/mL.

Embodiment 54: The method of any of embodiments 29 to 53, wherein the method further comprises a step of cleaving a plurality of nucleic acids with a non-specific nuclease after detection of the amplification threshold and the CRISPR threshold thereby reducing nucleic acid contamination in the system.

Embodiment 55: A method for detecting a target nucleic acid sequence comprising:
(a) adding a sample into a device comprising:
   an assay tube subdivided into a plurality of subsections, whereby a first subsection is connected to an openable cap, whereby an opening accessible from the openable cap in the first subsection is adapted for receiving a sample, whereby the first subsection is sealed from a subsequent subsection along the tube;
   a second subsection comprising a solution with internal control reagents, whereby the second subsection is sealed from a subsequent subsection along the tube;
   a third subsection comprising a solution with magnetic beads in solution, whereby the third subsection is sealed from a subsequent subsection along the tube;
   a fourth subsection comprising a solution with a lysis buffer, whereby the fourth subsection is sealed from a subsequent subsection along the tube;
   a fifth subsection comprising a solution with a wash buffer, whereby the fifth subsection is sealed from a subsequent subsection along the tube;
   a sixth subsection comprising a solution with an elution buffer, whereby the sixth subsection is sealed from a subsequent subsection along the tube;
   a seventh subsection comprising a solution with a PCR master mix part 1, whereby the seventh subsection is sealed from a subsequent subsection along the tube;
   an eighth subsection comprising a solution with a PCR master mix part 2, whereby the eighth subsection is sealed from a subsequent subsection along the tube; and
   a ninth tube comprising a solution with a CRISPR enzyme, whereby the ninth subsection is sealed from an end of the tube.
(b) adding the device comprising the sample into a system that supports a sequential rupture of one subsection from a subsequent subsection, thereby allowing the reagents in one subsection to sequentially react with the reagents in the subsequent subsection; and
(c) detecting the target nucleic acid by detecting a signal from the last subsection.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1A illustrates a device comprising an assay tube (elongated shape) subdivided into a plurality of subsections, with certain subsections having reagents for performing a nucleic acid amplification test;

FIG. 1B illustrates a device comprising an assay tube (elongated shape) subdivided into a plurality of subsections, with certain subsections having reagents for performing a nucleic acid amplification test and a CRISPR assay;

FIG. 2 illustrates a device of the disclosure along a system for providing a readout of a signal produced by a nucleic acid amplification test or a CRISPR assay;

FIG. 3 illustrates a device of the disclosure along a grading adapted for facilitating a sliding of the device into a system that provides a signal readout;

FIG. 4 is a schematic illustrating three distinct stages of a method of the disclosure for coupling signals detected from NAAT systems and CRISPR systems into one integrated process. Stage 1 illustrates the NAAT process with NAAT signal readout. Stage 2 illustrates the liquid handling process to enable amplicon transfer and interact with CRISPR systems. Stage 3 illustrates the CRISPR process with CRISPR signal readout;

FIG. 5 is a chart illustrating the results from experiments detecting a series of single sequence mutations in reference to a wild-type FluA sequence;

FIG. 6A-6D are charts illustrating: (A) generalized linear model fits detected in the CRISPR channel (Cas), along with its inverse prediction (B); and (C) generalized linear model fits detected in the CRISPR channel (Cas), along with its inverse prediction (D); and

FIG. 7 is a chart illustrating results of a CRISPR variation test.

It should be understood that the drawings are not necessarily to scale, and that like reference numbers refer to like features.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Terminology

As used herein, terms such as “first,” “second,” “third,” “fourth,” “fifth,” “sixth,” “seventh,” “eighth,” “ninth,” “tenth,” “n^{th},” or the like, merely identify one of a number of subsections, components, steps, operations, functions, and/or points of reference as disclosed herein, and likewise do not necessarily limit embodiments of the present disclosure to any particular configuration or orientation. Furthermore, terms such as “preceding,” “subsequent,” “left,” “right,” “top,” “bottom,” “front,” “rear,” “side,” “height,” “length,” “width,” “upper,” “lower,” “interior,” “exterior,” “inner,” and “outer” that may be used herein merely describe points of reference and do not necessarily limit embodiments of the present disclosure to any particular orientation or configuration.

The terms “target nucleic acid sequence” or “target sequence” and the like refer to any locus of a microorganism or population of microorganisms targeted for cleaving or amplification by a CRISPR system or a Nucleic Acid Amplification Test (NAAT) system.

As used herein the abbreviation “NAAT” generally refers to Nucleic Acid Amplification Tests.

As used herein the term “Ct” or “ct” refers to “cycle threshold” and is defined as the number of cycles required for a fluorescent signal to cross the threshold (i.e., exceeds background level). Ct levels are generally inversely proportional to the amount of target nucleic acid in the sample (i.e., the lower the Ct level the greater the amount of target nucleic acid in the sample). In a real time PCR assay a positive reaction is detected by accumulation of a fluorescent signal. The real time assays generally undergo 40 cycles of amplification. Cts<29 are strong positive reactions indicative of abundant target nucleic acid in the sample. Cts of 30-37 are positive reactions indicative of moderate amounts of target nucleic acid Cts of 38-40 are weak reactions indicative of minimal amounts of target nucleic acid which could represent an infection state or environmental contamination.

As used herein the abbreviation “CRISPR” generally refers to Clustered Regularly Interspaced Short Palindromic Repeats. Although there are diverse CRISPR-Cas systems among the different species of archaea and bacteria, these systems are generally connected by their dependence on a single-stranded RNA molecule (crRNA), which guides CRISPR proteins to recognize and cleave nucleic acid targets. The crRNA can be programmed towards a specific DNA or RNA region of interest through hybridization to a complementary sequence, which in some systems is restricted to the proximity of a protospacer adjacent motif (PAM) or protospacer flanking sequence.

As used herein the abbreviation “RSV” generally refers to Respiratory Syncytial Virus (RSV).

As used herein the abbreviations “FluA” and “FluB” generally refer to Influenza A and Influenza B, the two main types of influenza viruses that cause seasonal flu outbreaks. As used herein the term “Flu” refers to all viruses in the Influenza family.

As used herein the abbreviations “SARS-CoV-2” refers to the virus causing COVID-19.

As used herein, certain terms may have the following defined meanings. As used in the specification and claims, the singular form “a,” “an” and “the” include singular and plural references unless the context clearly dictates otherwise. For example, the term “a nucleic acid” includes a single nucleic acid molecule as well as a plurality of nucleic acids, including mixtures thereof.

As used herein, “about” means the recited quantity exactly and small variations within a limited range encompassing plus or minus 10% of the recited quantity. In other words, the limited range encompassed can include ±10%, ±9%, ±8%, ±7%, ±6%, ±5%, ±4%, ±3%, ±2%, ±1%, ±0.5%, ±0.2%, ±0.1%, ±0.05%, or smaller, as well as the recited value itself. Thus, by way of example, “about 10” should be understood to mean “10” and a range no larger than “9-11.”

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “percent sequence identity” with respect to a reference nucleic acid sequence is the percentage of nucleic acid bases in a target sequence that are identical with the nucleic acid bases in the reference sequence, respectively, after aligning the sequences and introducing gaps, if necessary, to achieve the maximum percent sequence identity. Methods of sequence alignment are well known in the art. Optimal alignment of sequences can be conducted by methods described in Needleman and Wunsch, 1970, J. Mol. Biol. 48:443; Pearson and Lipman, 1988, PNAS 85:2444, by computerized implementations of these algorithms. Alignments can be made using publicly available computer software such as BLASTp, BLASTn, BLAST-2, ALIGN or MegAlign Pro (DNASTAR) software.

As used herein the term “sample,” generally refers to any source of nucleic acids—either from a specimen, from a subject “hosting” the specimen or both—that can be informative of an environment. It may refer to samples derived from a subject, such as a nasal swab, blood, plasma, urine, tissue, faces, bone marrow, saliva, cerebrospinal fluid, or any other suitable tissue sample. It may refer to swab samples that are collected from surfaces in food processing facilities, long-term care facilities, hospitals, restaurants, or any suitable surface comprising nucleic acids. It may refer to a sample that comprises a biological tissue, soil, water, air, air filter materials, animal production, feed, manure, crop production, manufacturing plants, or any other suitable samples. Such samples may be derived from a hospital or a clinic and they may be analyzed on a mobile platform.

As used herein, the term “subject,” can refer to a human or to another animal. An animal can be a mouse, a rat, a guinea pig, a dog, a cat, a horse, a rabbit, and various other animals. A subject can be of any age, for example, a subject can be an infant, a toddler, a child, a pre-adolescent, an adolescent, an adult, or an elderly individual.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

In the following description, numerous specific details are set forth to provide a more thorough understanding of this disclosure. However, it will be apparent to one of skill in the art that this disclosure may be practiced without one or more of these specific details. In other instances, features and procedures well known to those skilled in the art have not been described in order to avoid obscuring this disclosure. The terms used herein are intended to have the plain and ordinary meaning as understood by those of ordinary skill in the art.

Nucleic acid amplification tests (NAAT) are widely used testing methods for infectious microorganisms across the globe, including SARS-CoV-2, FluA, FluB, and RSV. The most well-known and widely used NAAT is the polymerase chain reaction (PCR) and/or the reverse transcription polymerase chain reaction (RT-PCR). However, several other NAATs have been developed over the years, each with its own unique features and applications. Generally, however most NAAT technologies rely on the principle that a nucleic acid sequence with a certain complementarity to a target region can be used to amplify a target. Consequently, NAATs typically produce series of amplicons of sequences that were specifically targeted for amplification.

In contrast to NAATs, CRISPR systems generally consists of two main components: a ribonucleotide (RNA) sequence that carries a sequence complementarity to a target DNA and a CRISPR enzyme that functions along with the RNA sequence for performing a gene-editing function. In biotechnology, most CRISPR systems deploy a guide RNA (gRNA; which is either a “crRNA” or a “gRNA”) as single molecule that serves as a guide to direct a CRISPR enzyme (e.g., Cas9, Cas12) to a specific target DNA sequence, usually for gene editing or gene detection. Generally, one region of the gRNA contains a sequence that is complementary to the target sequence of interest, which is typically a target sequence adjacent to a Protospacer Adjacent Motif (PAM). Another target region acts as a scaffold that helps stabilize the complex between the gRNA and the CRISPR enzyme. It interacts with both the gRNA and the CRISPR enzyme, forming a stable ribonucleoprotein complex that guides the CRISPR enzyme to the target DNA for cleaving the target.

Thus, while NAAT approaches produce amplicons of a target region, when deployed for diagnostics, CRISPR methods largely tend to cleave a target sequence. The present disclosure provides novel devices, systems, and methods that integrate the outputs of NAAT and CRISPR assays to increase the specificity and inclusivity of target nucleic acid detection.

### Devices

Non-optimal oligo design or challenging reaction conditions could lead to false positive (specificity concern) or false negative (inclusivity concern) signal(s) in either NAAT or CRISPR strategies for detecting a microorganism(s). This can lead to the misidentification of target pathogens. Due to the difference in the target recognition mechanism of the two systems, the dual NAAT/CRISPR nature of the devices described herein virtually eliminate the possibility of a system doubly detecting a faulty signal. The present disclosure provides NAAT+CRISPR systems that improve the specificity and inclusivity of assay for target microorganism detection.

Devices of the disclosure generally comprise a plurality of subsections. A device of the disclosure can have, for example, one subsection, two subsections, three subsections, four subsections, five subsections, six subsections, seven subsections, eight subsections, nine subsections, ten subsections, eleven subsections, twelve subsections, thirteen subsections, fourteen subsections, fifteen subsections, sixteen subsections, seventeen subsections, eighteen subsections, nineteen subsections, twenty subsections, or another suitable number of subsections. In some configurations, a plurality of subsections are hermetic sealed with certain reagents for sample preparation and targeted analyte isolation, purification, extraction, target nucleic acid amplification, and target nucleic acid cleavage.

In some configurations, an elongated assay tube of the disclosure has a length of 70±20 millimeters, 71±20 millimeters, 72±20 millimeters, 73±20 millimeters, 74±20 millimeters, 75±20 millimeters, 76±20 millimeters, 77±20 millimeters, 78±20 millimeters, 79±20 millimeters, 80±20 millimeters, 81±20 millimeters, 82±20 millimeters, 83±20 millimeters, 84±20 millimeters, 85±20 millimeters, 86±20 millimeters, 87±20 millimeters, 88±20 millimeters, 89±20 millimeters, 90±20 millimeters, 91±20 millimeters, 92±20 millimeters, 93±20 millimeters, 94±20 millimeters, 95±20 millimeters, 96±20 millimeters, 97±20 millimeters, 98±20 millimeters, 99±20 millimeters, 100±20 millimeters, 101±20 millimeters, 102±20 millimeters, 103±20 millimeters, 104±20 millimeters, 105±20 millimeters, 106±20 millimeters, 107±20 millimeters, 108±20 millimeters, 109±20 millimeters, 110±20 millimeters, 111±20 millimeters, 112±20 millimeters, 113±20 millimeters, 114±20 millimeters, 115±20 millimeters, 116±20 millimeters, 117±20 millimeters, 118±20 millimeters, 119±20 millimeters, 120±20 millimeters, 121±20 millimeters, 122±20 millimeters, 123±20 millimeters, 124±20 millimeters, 125±20 millimeters, 126±20 millimeters, 127±20 millimeters, 128±20 millimeters, 129±20 millimeters, 130±20 millimeters, 131±20 millimeters, 132±20 millimeters, 133±20 millimeters, 134±20 millimeters, 135±20 millimeters, 136±20 millimeters, 137±20 millimeters, 138±20 millimeters, 139±20 millimeters, 140±20 millimeters, 70±21 millimeters, 71±21 millimeters, 72±21 millimeters, 73±21 millimeters, 74±21 millimeters, 75±21 millimeters, 76±21 millimeters, 77±21 millimeters, 78±21 millimeters, 79±21 millimeters, 80±21 millimeters, 81±21 millimeters, 82±21 millimeters, 83±21 millimeters, 84±21 millimeters, 85±21 millimeters, 86±21 millimeters, 87±21 millimeters, 88±21 millimeters, 89±21 millimeters, 90±21 millimeters, 91±21 millimeters, 92±21 millimeters, 93±21 millimeters, 94±21 millimeters, 95±21 millimeters, 96±21 millimeters, 97±21 millimeters, 98±21 millimeters, 99±21 millimeters, 100±21 millimeters, 101±21 millimeters, 102±21 millimeters, 103±21 millimeters, 104±21 millimeters, 105±21 millimeters, 106±21 millimeters, 107±21 millimeters, 108±21 millimeters, 109±21 millimeters, 110±21 millimeters, 111±21 millimeters, 112±21 millimeters, 113±21 millimeters, 114±21 millimeters, 115±21 millimeters, 116±21 millimeters, 117±21 millimeters, 118±21 millimeters, 119±21 millimeters, 120±21 millimeters, 121±21 millimeters, 122±21 millimeters, 123±21 millimeters, 124±21 millimeters, 125±21 millimeters, 126±21 millimeters, 127±21 millimeters, 128±21 millimeters, 129±21 millimeters, 130±21 millimeters, 131±21 millimeters, 132±21 millimeters, 133±21 millimeters, 134±21 millimeters, 135±21 millimeters, 136±21 millimeters, 137±21 millimeters, 138±21 millimeters, 139±21 millimeters, 140±21 millimeters, 70±22 millimeters, 71±22 millimeters, 72±22 millimeters, 73±22 millimeters, 74±22 millimeters, 75±22 millimeters, 76±22 millimeters, 77±22 millimeters, 78±22 millimeters, 79±22 millimeters, 80±22 millimeters, 81±22 millimeters, 82±22 millimeters, 83±22 millimeters, 84±22 millimeters, 85±22 millimeters, 86±22 millimeters, 87±22 millimeters, 88±22 millimeters, 89±22 millimeters, 90±22 millimeters, 91±22 millimeters, 92±22 millimeters, 93±22 millimeters, 94±22 millimeters, 95±22 millimeters, 96±22 millimeters, 97±22 millimeters, 98±22 millimeters, 99±22 millimeters, 100±22 millimeters, 101±22 millimeters, 102±22 millimeters, 103±22 millimeters, 104±22 millimeters, 105±22 millimeters, 106±22 millimeters, 107±22 millimeters, 108±22 millimeters, 109±22 millimeters, 110±22 millimeters, 111±22 millimeters, 112±22 millimeters, 113±22 millimeters, 114±22 millimeters, 115±22 millimeters, 116±22 millimeters, 117±22 millimeters, 118±22 millimeters, 119±22 millimeters, 120±22 millimeters, 121±22 millimeters, 122±22 millimeters, 123±22 millimeters, 124±22 millimeters, 125±22 millimeters, 126±22 millimeters, 127±22 millimeters, 128±22 millimeters, 129±22 millimeters, 130±22 millimeters, 131±22 millimeters, 132±22 millimeters, 133±22 millimeters, 134±22 millimeters, 135±22 millimeters, 136±22 millimeters, 137±22 millimeters, 138±22 millimeters, 139±22 millimeters, 140±22 millimeters, or another suitable length.

In some configurations, an elongated assay tube of the disclosure has a width of 2±1 millimeters, 3±1 millimeters, 4±1 millimeters, 5±1 millimeters, 6±1 millimeters, 7±1 millimeters, 8±1 millimeters, 9±1 millimeters, 10±1 millimeters, 11±1 millimeters, 12±1 millimeters, 13±1 millimeters, 14±1 millimeters, 15±1 millimeters, 16±1 millimeters, 17±1 millimeters, 18±1 millimeters, 19±1 millimeters, 20±1 millimeters, or another suitable width, or another suitable width.

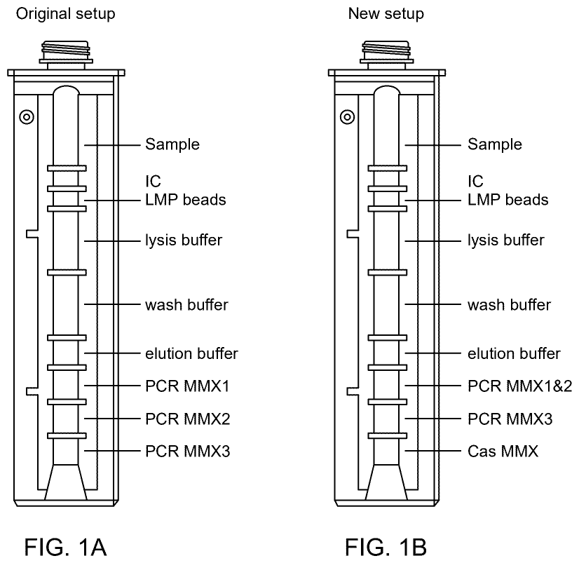

In most configurations, the length of each subsection is determined by the amount of reagents (volume) required for each step of the reaction. In some configurations each subsection in the plurality of subsections of the device can have a different length, as necessary for fitting the reagents required for each subsection. In some configurations two or more subsections, three or more subsections, four or more subsections, five or more subsections, six or more subsections, seven or more subsections, eight or more subsections, nine or more subsections, ten or more subsections can have a same length or a different length. See, e.g., FIGS. 1A, 1, 2, and 3 illustrating exemplary lengths of subsections in the device.

In some configurations the device is manufactured from a polymer. Non-limiting examples of polymers contemplated by the disclosure for manufacture of the device include thermoplastic polymers, as thermoplastic polymers can be repeatedly heated during thermocycling reaction steps without undergoing any significant chemical change. Examples of thermoplastic polymers contemplated by the disclosure include polyethylene (PE), including High-Density Polyethylene (HDPE) and Low-Density Polyethylene (LDPE); polypropylene (PP); polyvinyl chloride (PVC); polystyrene (PS); polyethylene terephthalate (PET); polyamide (Nylon); polycarbonate (PC); acrylonitrile butadiene styrene (ABS); poly(methyl methacrylate) (PMMA); or polyvinylidene fluoride (PVDF): PVDF.

In some configurations, devices of the disclosure provide reagents for dual-detection of one or more target microorganisms by combining NAAT strategies with CRISPR. One or more segments of a device of the disclosure typically comprises reagents for one or more of sample preparation and targeted analyte isolation, purification, extraction, target nucleic acid amplification, and target nucleic acid cleavage; depending on the NAAT technology selected for amplification and/or the CRISPR system selected for cleavage.

In certain configurations of the device, the first subsection is adapted for receiving a sample, such as saliva or sputum. The first subsection most typically comprises no reagents and is accessible via a cap, e.g., a screw on cap or a pressure type of a lid. The first subsection is generally adapted to receive a volume of sample to be analyzed in the assays. In certain configurations, a first subsection is adapted to receive a certain volume of sample. Non-limiting examples of volumes of sample include 50±40 microliters, 55±40 microliters, 60±40 microliters, 65±40 microliters, 70±40 microliters, 75±40 microliters, 80±40 microliters, 85±40 microliters, 90±40 microliters, 95±40 microliters, 100±40 microliters, 105±40 microliters, 110±40 microliters, 115±40 microliters, 120±40 microliters, 125±40 microliters, 130±40 microliters, 135±40 microliters, 140±40 microliters, 145±40 microliters, 150±40 microliters, 155±40 microliters, 160±40 microliters, 165±40 microliters, 170±40 microliters, 175±40 microliters, 180±40 microliters, 185±40 microliters, 190±40 microliters, 195±40 microliters, 200±40 microliters, 205±40 microliters, 210±40 microliters, 215±40 microliters, 220±40 microliters, 225±40 microliters, 230±40 microliters, 235±40 microliters, 240±40 microliters, 245±40 microliters, 250±40 microliters, 255±40 microliters, 260±40 microliters, 265±40 microliters, 270±40 microliters, 275±40 microliters, 280±40 microliters, 285±40 microliters, 290±40 microliters, 295±40 microliters, 300±40 microliters, 305±40 microliters, 310±40 microliters, 315±40 microliters, 320±40 microliters, 325±40 microliters, 330±40 microliters, 335±40 microliters, 340±40 microliters, 345±40 microliters, 350±40 microliters of sample, or another suitable volume of sample.

In certain configurations of the device, the second subsection is typically adapted for providing internal control reagents, such as positive or a negative control. A positive control can be, for example, a known sequence of a known target nucleic acid. It will become apparent that although in many configurations of device the internal control is placed in the second subsection, it is conceivable that the internal control could be placed on alternative subsections. In many configurations of the device, a subsection comprising the internal control has a volume of 5±2.5 microliters, 6±2.5 microliters, 7±2.5 microliters, 8±2.5 microliters, 9±2.5 microliters, 10±2.5 microliters, 11±2.5 microliters, 12±2.5 microliters, 13±2.5 microliters, 14±2.5 microliters, 15±2.5 microliters, 16±2.5 microliters, 17±2.5 microliters, 18±2.5 microliters, 19±2.5 microliters, 20±2.5 microliters, 21±2.5 microliters, 22±2.5 microliters, 23±2.5 microliters, 24±2.5 microliters, 25±2.5 microliters, or another suitable volume.

In certain configurations of the device, the third subsection is typically adapted for providing reagents for sample preparation and targeted analyte isolation, purification, and extraction. In many configurations, the third subsection is adapted for providing sorptive extraction techniques, i.e., magnetic separation or size separation for isolating a target nucleic acid from a sample, either through intrinsic characteristics of the sample or by labelling with magnetic materials. In certain configurations, the disclosure contemplates sorptive extraction techniques utilizing magnetic particles consisting of oxides of iron (e.g., magnetite or maghemite), nickel, and cobalt, or other elements combining several metals, such as zinc, copper, strontium, and barium. The magnetic particles can be of various shapes, including, but not limited to, spherical particles with a narrow size distribution, prepared by ball milling, co-precipitation, hydrothermal synthesis, thermal decomposition, laser ablation, microemulsion, chemical vapor deposition, arc discharge methods, flame spray synthesis, and biosynthesis methods. In some configurations, the magnetic particles contemplated by the disclosure can be functionalized with different compounds in order to provide functional groups for further bioactive molecule conjugations through different techniques, such as direct binding, Hong's method, or bioremediation. Specifically, the functionalization of magnetic particles allows for the attachment of ligands and receptors on their surface and the binding of biomolecules, such as monoclonal antibodies, nucleic acids, streptavidin, proteins, and peptides, to ensure specific interactions with the target molecules.

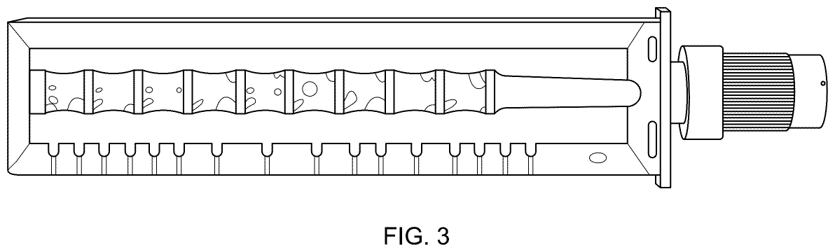

In some configurations, the disclosure contemplates one or more subsections comprising magnetic particles for selectively capturing, concentrating, transferring, and/or labelling targeted analytes, performing stringency and washing steps of target analytes (target nucleic acid sequences). In many instances the target nucleic acid sequence is a FluA, FluB, an RSV, or a SARS-CoV-2 target sequence. In the case of magnetic separation, the sample is allowed to come into contact with the magnetic particles, e.g., by sequentially exposing the sample to a series of reagents in the plurality of subsections of the assay tube—and incubated for a certain amount of time, to allow for the interaction with the target analytes through affinity adsorptions and/or antibody-antigen or hydrophobic interactions. As nucleic acids are polyanionic molecules with numerous phosphate groups, the electrostatic interactions could be increased through functionalization with positively charged species, such as aminosilanes; similarly, the immobilization of specific oligonucleotide sequences could allow for the affinity capture of nucleic acids through the hybridization of complementary sequences. Subsequently, the particles can separated from the sample by applying a magnet to the outside of the vessel wall (see, e.g., FIG. 3 illustrating an analysis system that can apply such magnetic field to an assay tube of the disclosure). The resulting analytes are eluted from the magnetic particles and subjected to further analyses. In some configurations of the device, a subsection comprising the magnetic particles has a volume of 5±2.5 microliters, 6±2.5 microliters, 7±2.5 microliters, 8±2.5 microliters, 9±2.5 microliters, 10±2.5 microliters, 11±2.5 microliters, 12±2.5 microliters, 13±2.5 microliters, 14±2.5 microliters, 15±2.5 microliters, 16±2.5 microliters, 17±2.5 microliters, 18±2.5 microliters, 19±2.5 microliters, 20±2.5 microliters, 21±2.5 microliters, 22±2.5 microliters, 23±2.5 microliters, 24±2.5 microliters, 25±2.5 microliters of a solution comprising a magnet, or another suitable volume.

In some configurations, the disclosure contemplates one or more subsections comprising a lysis buffer for, e.g., rupturing membranes of a cell or tissue. The specific composition of lysis buffer can vary significantly depending, e.g., on the sample to be analyzed, reagents compatible with an NAAT reaction, or reagents compatible with a CRISPR reaction. In many instances, the disclosure contemplates lysis buffers that include detergents, salts, buffering agents, protease inhibitors, and/or reducing agents. Non-limiting examples of detergents include triton X-100, NP-40, or tween 20. Non-limiting examples of salts include sodium chloride (NaCl) is a commonly used salt. Non-limiting examples of buffering agents include Tris-HCl or phosphate buffer. Non-limiting examples of protease inhibitors include PMSF (phenylmethylsulfonyl fluoride), aprotinin, or EDTA. Non-limiting examples of reducing agents include dithiothreitol (DTT) or 2-mercaptoethanol. In some configurations of the device, a subsection comprising the lysis buffer comprises 50±43 microliters, 55±43 microliters, 60±43 microliters, 65±43 microliters, 70±43 microliters, 75±43 microliters, 80±43 microliters, 85±43 microliters, 90±40 microliters, 95±43 microliters, 100±43 microliters, 105±43 microliters, 110±43 microliters, 115±43 microliters, 120±43 microliters, 125±43 microliters, 130±43 microliters, 135±43 microliters, 140±43 microliters, 145±43 microliters, 150±43 microliters, 155±43 microliters, 160±43 microliters, 165±43 microliters, 170±43 microliters, 175±43 microliters, 180±43 microliters, 185±43 microliters, 190±43 microliters, 195±43 microliters, 200±43 microliters, 205±43 microliters, 210±43 microliters, 215±43 microliters, 220±43 microliters, 225±43 microliters, 230±43 microliters, 235±43 microliters, 240±43 microliters, 245±43 microliters, 250±43 microliters, 255±43 microliters, 260±43 microliters, 265±43 microliters, 270±43 microliters, 275±43 microliters, 280±43 microliters, 285±43 microliters, 290±43 microliters, 295±43 microliters, 300±43 microliters, 305±43 microliters, 310±43 microliters, 315±43 microliters, 320±43 microliters, 325±43 microliters, 330±43 microliters, 335±43 microliters, 340±43 microliters, 345±43 microliters, 350±43 microliters of lysis buffer, or another suitable volume of a lysis buffer.

In some configurations, the disclosure contemplates one or more subsections comprising a wash buffer for removing unbound or nonspecifically bound substances while retaining the target molecules of interest. The specific composition of the wash buffer can vary significantly depending, e.g., on the sample to be analyzed, reagents compatible with an NAAT reaction, or reagents compatible with a CRISPR reaction. In many instances, the disclosure contemplates wash buffers that include one or more of buffering agents, salts, detergents, blocking agents, and/or other additives, e.g., chelating agents, reducing agents, and/or stabilizers. Non-limiting examples of buffering agents include tris-HCl. Non-limiting examples of salts include NaCl. Non-limiting examples of detergents include tween 20 and triton X-100. Non-limiting examples of blocking agents include bovine serum albumin (BSA). The number of segments comprising a wash buffer on a device can vary, e.g., depending on the target analyte to be analyzed. The number of segments comprising a wash buffer on the device itself and the washing conditions deployed in the method (e.g., incubation time, temperature, and agitation) can vary depending on the sensitivity of the target molecules. In some configurations of the device, a subsection comprising a wash buffer comprises 100±48 microliters, 105±48 microliters, 110±48 microliters, 115±48 microliters, 120±48 microliters, 125±48 microliters, 130±48 microliters, 135±48 microliters, 140±48 microliters, 145±48 microliters, 150±48 microliters, 155±48 microliters, 160±48 microliters, 165±48 microliters, 170±48 microliters, 175±48 microliters, 180±48 microliters, 185±48 microliters, 190±48 microliters, 195±48 microliters, 200±48 microliters, 205±48 microliters, 210±48 microliters, 215±48 microliters, 220±48 microliters, 225±48 microliters, 230±48 microliters, 235±48 microliters, 240±48 microliters, 245±48 microliters, 250±48 microliters, 255±48 microliters, 260±48 microliters, 265±48 microliters, 270±48 microliters, 275±48 microliters, 280±48 microliters, 285±48 microliters, 290±48 microliters, 295±48 microliters, 300±48 microliters, 305±48 microliters, 310±48 microliters, 315±48 microliters, 320±48 microliters, 325±48 microliters, 330±48 microliters, 335±48 microliters, 340±48 microliters, 345±48 microliters, 350±48 microliters, 355±48 microliters, 360±48 microliters, 365±48 microliters, 370±48 microliters, 375±48 microliters, 380±48 microliters, 385±48 microliters, 390±48 microliters, 395±48 microliters, 400±48 microliters of wash buffer, or another suitable volume of a wash buffer.

In some configurations, the disclosure contemplates one or more subsections comprising an elution buffer for releasing or “eluting” a target nucleic acid of interest from a magnetic bead, a solid support, or another binding matrix. Elution is the process of removing the specifically bound nucleic acids from the stationary phase (magnetic, resin, or matrix) into the liquid phase (elution buffer). The specific composition of the elution buffer can vary significantly depending, e.g., on the sample to be analyzed, reagents compatible with an NAAT reaction, or reagents compatible with a CRISPR reaction. In many instances, the disclosure contemplates elution buffers that include one or more of a salt, a pH adjusted buffer, a competitive ligand, an organic solvent, and/or a chelating agent. In some configurations of the device, a subsection comprising an elution buffer comprises 20±10 microliters, 25±10 microliters, 30±10 microliters, 35±10 microliters, 40±10 microliters, 45±10 microliters, 50±10 microliters, 55±10 microliters, 60±10 microliters, 65±10 microliters, 70±10 microliters, 75±10 microliters, 80±10 microliters, 85±10 microliters, 90±10 microliters, 95±10 microliters, 100±10 microliters, 135±10 microliters, 105±10 microliters, 110±10 microliters, 115±10 microliters, 120±10 microliters of elution buffer, or another suitable volume.

### Nucleic Acid Amplification Tests (NAAT)

In preferred configurations, the disclosure contemplates one or more, two or more, or three or more subsections comprising reagents for performing a nucleic acid amplification reaction (i.e., NAAT). The disclosure contemplates a variety of mechanistically distinct strategies for amplifying a target nucleic acid, and in some aspects, the disclosure contemplates combinations of strategies where one or more segments of the device includes reagents for a polymerase chain reaction (PCR), a reverse-transcription polymerase chain reaction (RT-PCR), a loop mediated isothermal amplification (LAMP) reaction, a recombinase polymerase amplification (RPA) reaction, a nucleic acid sequence based amplification (NASBS) reaction, a transcription mediated amplification (TMA) reaction, a rolling circle amplification (RCA) reaction, a strand displacement amplification (SDA) reaction, a nicking and extension amplification reaction (NEAR), an exponential amplification reaction (EXPAR), a multiple displacement amplification (MDA) reaction, a helicase dependent amplification (HAD) reaction, or a hybridization chain reaction (HCR).

In some configurations the nucleic acid amplification reaction is a polymerase chain reaction (PCR), e.g., an RT-PCR (Reverse Transcription Polymerase Chain Reaction). Amplification of nucleic acid sequences by PCR is described in U.S. Pat. Nos. 4,683,195, 4,683,202, and 4,965,188. PCR is now well known in the art and has been described extensively in the scientific literature. See PCR Applications, ((1999) Innis et al., eds., Academic Press, San Diego), PCR Strategies, ((1995) Innis et al., eds., Academic Press, San Diego); PCR Protocols, ((1990) Innis et al., eds., Academic Press, San Diego), and PCR Technology, ((1989) Erlich, ed., Stockton Press, New York). A “real-time” PCR assay is able to simultaneously amplify and detect and/or quantify the starting amount of the target sequence. The basic TaqMan real-time PCR assay using the 5′-to-3′ nuclease activity of the DNA polymerase is described in Holland et al., (1991) Proc. Natl. Acad. Sci. 88:7276-7280 and U.S. Pat. No. 5,210,015. A real-time PCR without the nuclease activity (a nuclease-free assay) has been described in U.S. Patent Publication No. 20100143901A1. The use of fluorescent probes in real time PCR is described in U.S. Pat. No. 5,538,848. A rapid one-step reverse transcriptase PCR (RT-PCR) is described in U.S. Pat. No. 8,119,353B2.

In some configurations the nucleic acid amplification reaction is a loop mediated isothermal amplification (LAMP) reaction. LAMP approaches deploy multiple primers targeting several regions of the target DNA, thus resulting in high amplification efficiency of a target nucleic acid. LAMP can amplify DNA at a constant temperature, typically between 60° C. to 65° C. Amplification of nucleic acid sequences by LAMP is described in U.S. Pat. No. 6,410,278 and extensively in the literature. See, e.g., Notomi T, Okayama H, Masubuchi H, Yonekawa T, Watanabe K, Amino N, Hase T (2000). “Loop-mediated isothermal amplification of DNA”. Nucleic Acids Res. 28 (12): 63e-63; Shirshikov, Fedor V.; Pekov, Yuri A.; Miroshnikov, Konstantin A. (2019 Apr. 26). “MorphoCatcher: a multiple-alignment based web tool for target selection and designing taxon-specific primers in the loop-mediated isothermal amplification method”. PeerJ. 7: e6801; Mori Y, Kitao M, Tomita N, Notomi T (2004). “Real-time turbidimetry of LAMP reaction for quantifying template DNA.” J. Biochem. Biophys. Methods. 59 (2): 145-57; Calvert, Amanda E.; Biggerstaff, Brad J.; Tanner, Nathan A.; Lauterbach, Molly; Lanciotti, Robert S. (2017). “Rapid colorimetric detection of Zika virus from serum and urine specimens by reverse transcription loop-mediated isothermal amplification (RT-LAMP).” PLOS ONE. 12 (9): e0185340.

In some configurations the nucleic acid amplification reaction is a nucleic acid sequence based amplification (NASBS) reaction for producing multiple copies of single stranded RNA. NASBS is a primer-dependent technology that can be used for the continuous amplification of nucleic acids in a single mixture at one or two temperatures. Amplification of nucleic acid sequences by NASBS is described in e.g., Deiman, Birgit; van Aarle, Pierre; Sillekens, Peter (2002). “Characteristics and Applications of Nucleic Acid Sequence-Based Amplification (NASBA)”. Molecular Biotechnology. 20 (2): 163-180; Malek, L.; Sooknanan, R.; Compton, J. (1994). Nucleic acid sequence-based amplification (NASBA). Methods in Molecular Biology. Vol. 28. pp. 253-260; Compton, J (1991). “Nucleic acid sequence-based amplification”. Nature. 350 (6313): 91-2.

In some configurations the nucleic acid amplification reaction is a transcription mediated amplification (TMA) reaction, an isothermal amplification system utilizing two enzymes, RNA polymerase and reverse transcriptase. Amplification of nucleic acid sequences by TMA is described in, e.g., Daniel L. Kacian, Timothy J. Fultz: Nucleic acid sequence amplification methods. In: Biotechnology Advances 1995, 13.3, S. 569-569.

In some configurations the nucleic acid amplification reaction is a rolling circle amplification (RCA) reaction. RCA is an isothermal nucleic acid amplification technique where the polymerase continuously adds single nucleotides to a primer annealed to a circular template which results in a long concatemer ssDNA that contains tens to hundreds of tandem repeats (complementary to the circular template). RCA is now well known in the art and has been described extensively in the scientific literature. See, e.g., Ali, M. Monsur; Li, Feng; Zhang, Zhiqing; Zhang, Kaixiang; Kang, Dong-Ku; Ankrum, James A.; Le, X. Chris; Zhao, Weian (2014). “Rolling circle amplification: a versatile tool for chemical biology, materials science and medicine”. Chemical Society Reviews. 43 (10): 3324-41; Lizardi, Paul M.; Huang, Xiaohua; Zhu, Zhengrong; Bray-Ward, Patricia; Thomas, David C.; Ward, David C. (July 1998). “Mutation detection and single-molecule counting using isothermal rolling-circle amplification”. Nature Genetics. 19 (3): 225-232.

In some configurations the nucleic acid amplification reaction is a strand displacement amplification (SDA) reaction or a multiple displacement amplification (MDA) reaction. SDA and MDA are an isothermal amplification technique based upon the ability of a nicking enzyme, e.g., HincII, to nick the unmodified strand of strand (e.g., a hemiphosphorothioate form of HincII recognition site), and the ability of exonuclease deficient klenow (exo-klenow) to extend the 3′-end at the nick and displace the downstream DNA strand. Exponential amplification results from coupling sense and antisense reactions in which strands displaced from a sense reaction serve as target for an antisense reaction and vice versa. SDA is now well known in the art. See, e.g., G. T. Walker, M. C. Little, J. G. Nadeau and D. D. Shank (1992) Proc. Natl. Acad. Sci 89, 392-396; Spits; Le Caignec, C; De Rycke, M; Van Haute, L; Van Steirteghem, A; Liebaers, I; Sermon, K (2006). “Whole-genome multiple displacement amplification from single cells”. Nature Protocols. 1 (4): 1965-70.

In some configurations the nucleic acid amplification reaction is a Nicking Enzyme Amplification Reaction (NEAR). NEAR is isothermal, replicating DNA at a constant temperature using a polymerase (and nicking enzyme) to exponentially amplify the DNA at a temperature range of 55° C. to 59° C. NEAR has been described in the literature in U.S. Pat. Nos. 6,191,267 and 6,660,475. NEAR is further described in the art in Biochemistry. 2008 Sep. 23; 47(38):9987-99.

In some configurations the nucleic acid amplification reaction is a helicase dependent amplification (HAD) reaction. HAD utilizes a DNA helicase to generate single-stranded templates for primer hybridization and subsequent primer extension by a DNA polymerase. HAD has been described in the literature in Saiki R K, et al. (1988). “Primer-directed enzymatic amplification of DNA with a thermostable DNA polymerase”. Science. 239 (4839): 487-491; and Kornberg A, Baker T (1992). DNA Replication, 2nd edn. WH Freeman and Company: New York.

In some configurations the nucleic acid amplification reaction is a hybridization chain reaction (HCR). HCR provides multiplexed, isothermal, enzyme-free, molecular signal amplification in diverse settings.

In preferred configurations, the disclosure contemplates one or more, two or more, or three or more subsections comprising reagents for performing a series of nucleic acid amplification reactions (i.e., NAAT). In some instances, one segment of the device comprises reagents for an RT-PCR reaction, e.g., reaction buffer, dNTP, RT-PCR primers (e.g., primers for amplifying one or more target sequences from, e.g., FluA, FluB, RSV, and/or SARS-CoV-2), a reverse polymerase (e.g., MMLV RT polymerase), and/or a Uracil-DNA glycosylase. In some instances, one segment of the device comprises reagents for a PCR reaction, e.g., reaction buffer, a polymerase and required functional components (Z05 polymerase and its aptamer), and/or RT-PCR primers. In some configurations of the device, a subsection comprising a NAAT reaction mix (“a NAAT master mix”) comprises 5±3 microliters, 10±3 microliters, 15±3 microliters, 20±3 microliters, 25±3 microliters, 30±3 microliters, 35±3 microliters, 40±3 microliters, 45±3 microliters, 50±3 microliters, 55±3 microliters, 60±3 microliters, 65±3 microliters, 70±3 microliters, 75±3 microliters, 80±3 microliters, 10±6 microliters, 15±6 microliters, 20±6 microliters, 25±6 microliters, 30±6 microliters, 35±6 microliters, 40±6 microliters, 45±6 microliters, 50±6 microliters, 55±6 microliters, 60±6 microliters, 65±6 microliters, 70±6 microliters, 75±6 microliters, 80±6 microliters of a NAAT master mix, or another suitable volume with the functional reagents for the NAAT methodology selected for manufacturing on the device.

### CRISPR Systems

Since their initial discovery, the number of different CRISPR-Cas systems has expanded rapidly. Currently, CRISPR-Cas systems can be divided, according to evolutionary relationships, into two classes, six types and several subtype. The classes of CRISPR-Cas system are defined by the nature of the ribonucleoprotein effector complex: class 1 systems are characterized by a complex of multiple effector proteins, and class 2 systems encompass a single crRNA-binding protein. The main difference between CRISPR type II (Cas9) systems and type V (Cas12) and type VI (Cas13) systems is the ability of the latter two systems to trigger non-specific collateral cleavage (trans cleavage) on target recognition. Collateral activity involves the cleavage of non-targeted single-stranded DNA (ssDNA; Cas12) or single-stranded RNA (ssRNA; Cas13) in solution, which enables the sensing of nucleic acids through signal amplification and allows for various readouts through the addition of functionalized reporter nucleic acids, which are generally cleaved by collateral activity.

In CRISPR-based diagnostics, quantification through comparison with a standard curve can be achieved within the picomolar-to-micromolar range (10⁻¹² M-10⁻⁶ M), where CRISPR-based collateral-cleavage activity correlates with target concentration.

In some configurations, the disclosure contemplates one or more, two or more, or three or more subsections comprising CRISPRs reagents (enzymes, guide RNAs, substrates, co-factors) with trans-cleavage activity. Upon activation by a target nucleic acid the CRISPR enzymes with trans-cleavage activity, (e.g., Cas12 and Cas13) indiscriminately cleave short single-stranded reporter oligos. When target nucleotides are labeled with reporter molecules, such cleavage separates the fluorophore from the quencher which are typically labeled at opposite ends of the reporter molecule, and generating measurable fluorescence signals.

In some configurations, the disclosed devices for NAAT+CRISPR dual detection system can be used to target different pathogens from the same patient sample input. Since the signals are generated at different stages (see, e.g., FIG. 3), two different pathogens can be readout from the same optical channel, therefore increasing the multiplexing capability of NAAT without the need to increase physical analyzer system setup (optical channels). Non-limiting examples of suitable analyzer systems and existing sample processing tubes of the art are described in U.S. Pat. Nos. 6,036,920; 7,718,421; 9,005,551; 9,708,599, and 10,443,050.

In some configurations of the device, a subsection comprising a CRISPR reaction mix (“a CRISPR master mix”) comprises 20±12 microliters, 25±12 microliters, 30±12 microliters, 35±12 microliters, 40±12 microliters, 45±12 microliters, 50±12 microliters, 55±12 microliters, 60±12 microliters, 65±12 microliters, 70±12 microliters, 75±12 microliters, 80±12 microliters, 10±12 microliters, 15±12 microliters, 20±12 microliters, 25±12 microliters, 30±12 microliters, 35±12 microliters, 40±12 microliters, 45±12 microliters, 50±12 microliters, 55±12 microliters, 60±12 microliters, 65±12 microliters, 70±12 microliters, 75±12 microliters, 80±12 microliters, 85±12 microliters, 90±12 microliters, 95±12 microliters, 100±12 microliters, 105±12 microliters, 110±12 microliters, 115±12 microliters, 120±12 microliters, of a CRISPR master mix, or another suitable volume with the functional reagents for the CRISPR enzyme selected for inclusion on the device.

### Methods

All of the functionalities described in connection with the devices, systems, and processes described herein are intended to be applicable to detection of at least one target microorganism from a sample. The sample can comprise a mixture of viral nucleic acids, mammalian nucleic acids, and bacterial nucleic acids.

Certain signals produced from nucleic acid amplification tests could be low and difficult to distinguish from negative control signal when the pathogen concentration in the sample is low or near detection limit. In certain aspects, the disclosure addresses this challenge by deploying a CRISPR system assay that can further amplify the amplicon signal from an NAAT with its collateral cleavage activity, i.e., one molecule of NAAT amplicon could generates multiple signal molecules (e.g., fluorescence labels). In contrast, in a device or analyzer system that solely detects a NAAT signal, one molecule of NAAT amplicon usually generates only one signal molecules. Therefore, the devices, systems, and methods of the disclosure, which integrate signals from NAAT and CRISPR systems can produce an increased true signal and improve robustness of an assay for detecting a microorganism.

In some implementations the disclosure provides a method for dual-detection of a target microorganism comprising: amplifying a first target nucleic acid sequence in a nucleic acid amplification reaction and detecting a signal from the nucleic acid amplification of the first target nucleic acid sequence; detecting a second target nucleic acid sequence with a CRISPR system targeting the second target nucleic acid sequence and detecting a signal from the second target nucleic acid activation and substrate cleavage by the CRISPR system; comparing the signal detected from the nucleic acid amplification of the first target nucleic acid sequence with an amplification threshold and comparing the signal detected from the second target nucleic acid activation and substrate cleavage by the CRISPR system with a CRISPR threshold; wherein the first target nucleic acid sequence and the second target nucleic acid sequence are sequences from the target microorganism; whereby the target nucleic acid sequence is dually-detected when the signal detected from the first target nucleic acid amplification reaction and the signal detected from the second target nucleic acid activation and substrate cleavage by the CRISPR system are both above the threshold. In some implementations the first target nucleic acid sequence is amplified with primers selected from the group consisting of SEQ ID NOs: 2-5. In some implementations the CRISPR system targeting the second target nucleic acid has a guide sequence of SEQ ID NO: 1.

In some implementations the disclosure provides a method of for detecting a target nucleic acid sequence comprising: (a) adding a sample into a device comprising: an assay tube subdivided into a plurality of subsections, whereby a first subsection is connected to an openable cap, whereby an opening accessible from the openable cap in the first subsection is adapted for receiving a sample, whereby the first subsection is sealed from a subsequent subsection along the assay tube; a second subsection comprising a solution with internal control reagents, whereby the second subsection is sealed from a subsequent subsection along the assay tube; a third subsection comprising a solution with magnetic beads in solution, whereby the third subsection is sealed from a subsequent subsection along the assay tube; a fourth subsection comprising a solution with a lysis buffer, whereby the fourth subsection is sealed from a subsequent subsection along the assay tube; a fifth subsection comprising a solution with a wash buffer, whereby the fifth subsection is sealed from a subsequent subsection along the assay tube; a sixth subsection comprising a solution with an elution buffer, whereby the sixth subsection is sealed from a subsequent subsection along the assay tube; a seventh subsection comprising a solution with a first PCR master mix (part 1), whereby the seventh subsection is sealed from a subsequent subsection along the assay tube; an eighth subsection comprising a solution with a second PCR master mix (part 2), whereby the eighth subsection is sealed from a subsequent subsection along the assay tube; and a ninth tube comprising a solution with a CRISPR enzyme, whereby the ninth subsection is sealed from an end of the assay tube; (b) adding the device comprising the sample into a system that supports a sequential rupture of one subsection from a subsequent subsection, thereby allowing the reagents in one subsection to sequentially react with the reagents in the subsequent subsection; and (b) detecting the target nucleic acid by detecting a signal from the last subsection. Non-limiting examples of suitable analyzer systems and methods for use with a device of the disclosure are described in U.S. Pat. Nos. 6,036,920; 6,036,920; 7,718,421; 8,936,933; 9,005,551; 9,708,599, and 10,443,050.

In certain implementations of this method, the devices described herein are configured for the distinguishing at least one microorganism(s) from a plurality of microorganisms selected from the group consisting of SARS-CoV-2, influenza A, influenza B, Human Respiratory Syncytial Virus (RSV). In other cases, at least one specimen in the plurality of nucleic acids is selected from the group consisting of SARS-CoV-2, influenza A, influenza B, Human Respiratory Syncytial Virus (RSV), adenovirus, coronavirus 229E, coronavirus HKU1, coronavirus NL63, human metapneumovirus, human rhinovirus/enterovirus, parainfluenza virus 1, parainfluenza virus 2, parainfluenza virus 3, parainfluenza virus 4.

In some configurations of the device, the device comprises one or more subsections with CRISPR enzymes having with trans-cleavage activity such as Cas12 and Cas13, guide RNA, substrates, metal ion co-factors for targeting a region of a target sequence (e.g., FluA, FluB, RSV, and/or SARS-CoV-2. In some implementations of the device, the device comprises additional subsections wherein each subsection comprises a set of CRISPR systems reagents for detecting one or more of FluA, FluB, RSV, and/or SARS-CoV-2. Non-limiting examples of microorganisms from the Corona genus that can be distinguished with the methods of the disclosure include both viruses with low case fatality rate (CFR, HCoV-NL63, HCoV-229E, HCoV-OC43, and HCoV-HKU1, and those with high CFR, namely, MERS-CoV, SARS-CoV, and SARS-CoV-2.

### Sequences

Table 1 describes non-limiting examples of guide RNA sequence(s), primers, and probes that can be deployed to detect Flu type A.

TABLE 1
FluA

| | SEQ. ID. | | |
|---|---|---|---|
| Name | NO: | Sequence | |
| Cas12 guide | 1 | UAAUUUCUACUAAGUGUAGAUGGAUUUGUGUUCACGCUCAC | |
| RNA | | | |
| FluA | 2 | CCAATCCTGTCACCTCTGACTAA | |
| forward | | | |
| primer 1 | | | |
| FluA | 3 | CCAATCCTGTCACCTCTGACCA | |
| forward | | | |
| primer 2 | | | |
| FluA | 4 | CCAATCCTGTCACCTTTGACTAA | |
| forward | | | |
| primer 3 | | | |
| FluA | 5 | GCGTCTACGCTGCAGTC | |
| reverse | | | |
| primer | | | |
| FluA probe | 6 | /5cy55/CTCACTGGGCACGGTGAGCG/3BHQ_2/ | |
| FluA probe | 7 | /5TYE705/CTCACTGGGCACGGTGAGCG/3BHQ_2/ | |
| alternative | | | |
| FluA target | 8 | CCAATCTTGTCACCTCTGACTAAGGGAATTTTAGGATTTGTGTTC | |
| sequence | | ACGCTCACCGTGCCCAGTGAGCGAGGACTGCAGCGTAGACGC | |
| | SEQ. ID. | | |
| Name | NO: | Sequence | Comments |
| FS-T-1 | 9 | CCAATCTTGTCACCTCTGACTAAGGGAATTTT | ssDNA target WT |
| | | AGGATTTGTGTTCACGCTCACCGTGCCCAGT | |
| | | GAGCGAGGACTGCAGCGTAGACGC | |
| FS-T-11 | 10 | CCAATCTTGTCACCTCTGACTAAGGGAATCTT | ssDNA target with |
| | | AGGATTTGTGTTCACGCTCACCGTGCCCAGT | single mismatch |
| | | GAGCGAGGACTGCAGCGTAGACGC | |
| FS-T-12 | 11 | CCAATCTTGTCACCTCTGACTAAGGGAATTCT | ssDNA target with |
| | | AGGATTTGTGTTCACGCTCACCGTGCCCAGT | single mismatch |
| | | GAGCGAGGACTGCAGCGTAGACGC | |
| FS-T-13 | 12 | CCAATCTTGTCACCTCTGACTAAGGGAATTT | ssDNA target with |
| | | AAGGATTTGTGTTCACGCTCACCGTGCCCAG | single mismatch |
| | | TGAGCGAGGACTGCAGCGTAGACGC | |
| FS-T-14 | 13 | CCAATCTTGTCACCTCTGACTAAGGGAATTTT | ssDNA target with |
| | | TGGATTTGTGTTCACGCTCACCGTGCCCAGTG | single mismatch |
| | | AGCGAGGACTGCAGCGTAGACGC | |
| FS-T-15 | 14 | CCAATCTTGTCACCTCTGACTAAGGGAATTTT | ssDNA target with |
| | | ACGATTTGTGTTCACGCTCACCGTGCCCAGT | single mismatch |
| | | GAGCGAGGACTGCAGCGTAGACGC | |
| FS-T-16 | 15 | CCAATCTTGTCACCTCTGACTAAGGGAATTTT | ssDNA target with |
| | | AGTATTTGTGTTCACGCTCACCGTGCCCAGTG | single mismatch |
| | | AGCGAGGACTGCAGCGTAGACGC | |
| FS-T-17 | 16 | CCAATCTTGTCACCTCTGACTAAGGGAATTTT | ssDNA target with |
| | | AGGCTTTGTGTTCACGCTCACCGTGCCCAGT | single mismatch |
| | | GAGCGAGGACTGCAGCGTAGACGC | |
| FS-T-18 | 17 | CCAATCTTGTCACCTCTGACTAAGGGAATTTT | ssDNA target with |
| | | AGGAATTGTGTTCACGCTCACCGTGCCCAGT | single mismatch |
| | | GAGCGAGGACTGCAGCGTAGACGC | |
| FS-T-19 | 18 | CCAATCTTGTCACCTCTGACTAAGGGAATTTT | ssDNA target with |
| | | AGGATGTGTGTTCACGCTCACCGTGCCCAGT | single mismatch |
| | | GAGCGAGGACTGCAGCGTAGACGC | |
| FS-T-20 | 19 | CCAATCTTGTCACCTCTGACTAAGGGAATTTT | ssDNA target with |
| | | AGGATTCGTGTTCACGCTCACCGTGCCCAGT | single mismatch |
| | | GAGCGAGGACTGCAGCGTAGACGC | |
| FS-T-21 | 20 | CCAATCTTGTCACCTCTGACTAAGGGAATTTT | ssDNA target with |
| | | AGGATTTTTGTTCACGCTCACCGTGCCCAGTG | single mismatch |
| | | AGCGAGGACTGCAGCGTAGACGC | |
| FS-T-22 | 21 | CCAATCTTGTCACCTCTGACTAAGGGAATTTT | ssDNA target with |
| | | AGGATTTGAGTTCACGCTCACCGTGCCCAGT | single mismatch |
| | | GAGCGAGGACTGCAGCGTAGACGC | |
| FS-T-23 | 22 | CCAATCTTGTCACCTCTGACTAAGGGAATTTT | ssDNA target with |
| | | AGGATTTGTATTCACGCTCACCGTGCCCAGT | single mismatch |
| | | GAGCGAGGACTGCAGCGTAGACGC | |
| FS-T-24 | 23 | CCAATCTTGTCACCTCTGACTAAGGGAATTTT | ssDNA target with |
| | | AGGATTTGTGATCACGCTCACCGTGCCCAGT | single mismatch |
| | | GAGCGAGGACTGCAGCGTAGACGC | |
| FS-T-25 | 24 | CCAATCTTGTCACCTCTGACTAAGGGAATTTT | ssDNA target with |
| | | AGGATTTGTGTCCACGCTCACCGTGCCCAGT | single mismatch |
| | | GAGCGAGGACTGCAGCGTAGACGC | |
| FS-T-26 | 25 | CCAATCTTGTCACCTCTGACTAAGGGAATTTT | ssDNA target with |
| | | AGGATTTGTGTTTACGCTCACCGTGCCCAGT | single mismatch |
| | | GAGCGAGGACTGCAGCGTAGACGC | |
| FS-T-27 | 26 | CCAATCTTGTCACCTCTGACTAAGGGAATTTT | ssDNA target with |
| | | AGGATTTGTGTTCCCGCTCACCGTGCCCAGT | single mismatch |
| | | GAGCGAGGACTGCAGCGTAGACGC | |
| FS-T-28 | 27 | CCAATCTTGTCACCTCTGACTAAGGGAATTTT | ssDNA target with |
| | | AGGATTTGTGTTCAGGCTCACCGTGCCCAGT | single mismatch |
| | | GAGCGAGGACTGCAGCGTAGACGC | |
| FS-T-29 | 28 | CCAATCTTGTCACCTCTGACTAAGGGAATTTT | ssDNA target with |
| | | AGGATTTGTGTTCACTCTCACCGTGCCCAGTG | single mismatch |
| | | AGCGAGGACTGCAGCGTAGACGC | |
| FS-T-30 | 29 | CCAATCTTGTCACCTCTGACTAAGGGAATTTT | ssDNA target with |
| | | AGGATTTGTGTTCACGATCACCGTGCCCAGT | single mismatch |
| | | GAGCGAGGACTGCAGCGTAGACGC | |
| FS-T-31 | 30 | CCAATCTTGTCACCTCTGACTAAGGGAATTTT | ssDNA target with |
| | | AGGATTTGTGTTCACGCCCACCGTGCCCAGT | single mismatch |
| | | GAGCGAGGACTGCAGCGTAGACGC | |
| FS-T-32 | 31 | CCAATCTTGTCACCTCTGACTAAGGGAATTTT | ssDNA target with |
| | | AGGATTTGTGTTCACGCTTACCGTGCCCAGT | single mismatch |
| | | GAGCGAGGACTGCAGCGTAGACGC | |
| FS-T-33 | 32 | CCAATCTTGTCACCTCTGACTAAGGGAATTTT | ssDNA target with |
| | | AGGATTTGTGTTCACGCTCGCCGTGCCCAGT | single mismatch |
| | | GAGCGAGGACTGCAGCGTAGACGC | |
| FS-T-34 | 33 | CCAATCTTGTCACCTCTGACTAAGGGAATTTT | ssDNA target with |
| | | AGGATTTGTGTTCACGCTCATCGTGCCCAGT | single mismatch |
| | | GAGCGAGGACTGCAGCGTAGACGC | |
| FS-T-35 | 34 | CCAATCTTGTCACCTCTGACTAAGGGAATTTT | ssDNA target with |
| | | AGGATTTGTGTTCACGCTCACAGTGCCCAGT | single mismatch |
| | | GAGCGAGGACTGCAGCGTAGACGC | |
| FS-T-36 | 35 | CCAATCTTGTCACCTCTGACTAAGGGAATTTT | ssDNA target with |
| | | AGGATTTGTGTTCACGCTCACCTTGCCCAGTG | single mismatch |
| | | AGCGAGGACTGCAGCGTAGACGC | |
| FS-T-37 | 36 | CCAATCTTGTCACCTCTGACTAAGGGAATTTT | ssDNA target with |
| | | AGGATTTGTGTTCACGCTCACCGGGCCCAGT | single mismatch |
| | | GAGCGAGGACTGCAGCGTAGACGC | |
| FS-T-38 | 37 | CCAATCTTGTCACCTCTGACTAAGGGAATTTT | ssDNA target with |
| | | AGGATTTGTGTTCACGCTCACCGTCCCCAGT | single mismatch |
| | | GAGCGAGGACTGCAGCGTAGACGC | |
| FS-T-39 | 38 | CCAATCTTGTCACCTCTGACTAAGGGAATTTT | ssDNA target with |
| | | AGGATTTGTGTTCACGCTCACCGTGACCAGT | single mismatch |
| | | GAGCGAGGACTGCAGCGTAGACGC | |
| FS-T-40 | 39 | CCAATCTTGTCACCTCTGACTAAGGGAATTTT | ssDNA target with |
| | | AGGATTTGTGTTCACGCTCACCGTGCGCAGT | single mismatch |
| | | GAGCGAGGACTGCAGCGTAGACGC | |
| FS-T-41 | 40 | CCAATCTTGTCACCTCTGACTAAGGGAATTTT | ssDNA target with |
| | | AGGATTTGTGTTCACGCTCACCGTGCCGAGT | single mismatch |
| | | GAGCGAGGACTGCAGCGTAGACGC | |
| FS-T-42 | 41 | CCAATCTTGTCACCTCTGACTAAGGGAATTTT | ssDNA target with |
| | | AGGATTTGTGTTCACGCTCACCGTGCCCTGTG | single mismatch |
| | | AGCGAGGACTGCAGCGTAGACGC | |
| FS-T-43 | 42 | CCAATCTTGTCACCTCTGACTAAGGGAATTTT | ssDNA target with |
| | | AGGATTTGTGTTCACGCTCACCGTGCCCACT | single mismatch |
| | | GAGCGAGGACTGCAGCGTAGACGC | |
| FS-T-44 | 43 | CCAATCTTGTCACCTCTGACTAAGGGAATTTT | ssDNA target with |
| | | AGGATTTGTGTTCACGCTCACCGTGCCCAGC | single mismatch |
| | | GAGCGAGGACTGCAGCGTAGACGC | |
| FS-T-45 | 44 | CCAATCTTGTCACCTCTGACTAAGGGAATTTT | ssDNA target with |
| | | AGGATTTGTGTTCACGCTCACCGTGCCCAGT | single mismatch |
| | | CAGCGAGGACTGCAGCGTAGACGC | |
| FS-T-46 | 45 | CCAATCTTGTCACCTCTGACTAAGGGAATTTT | ssDNA target with |
| | | AGGATTTGTGTTCACGCTCACCGTGCCCAGT | single mismatch |
| | | GTGCGAGGACTGCAGCGTAGACGC | |
| FS-T-47 | 46 | CCAATCTTGTCACCTCTGACTAAGGGAATTTT | ssDNA target with |
| | | AGGATTTGTGTTCACGCTCACCGTGCCCAGT | single mismatch |
| | | GACCGAGGACTGCAGCGTAGACGC | |

FluB

| | SEQ. ID. | | |
|---|---|---|---|
| Name | NO: | Sequence | Comments |
| FluB | 47 | CGAGCGTCTTAATGAAGGACA | FluB PCR oligos |
| forward | | | |
| primer | | | |
| FluB | 48 | TGCTCTTGACCAAATTGGGATA | FluB PCR oligos |
| reverse | | | |
| primer | | | |
| FluB probe | 49 | /5TEX615/AAGCCAATTCGAGCAGCTGAAACT | FluB PCR oligos |
| | | GCG/3BHQ_2/ | |
| LwaCas13 | 50 | GAUUUAGACUACCCCAAAAACGAAGGGGAC | LwaCas13 system |
| guide RNA | | UAAAACAGGACAUUCAAAGCCAAUUCGAGC | |
| 1 | | AGCU | |
| LwaCas13 | 51 | GAUUUAGACUACCCCAAAAACGAAGGGGAC | LwaCas13 system |
| guide RNA | | UAAAACAAAGCCAAUUCGAGCAGCUGAAAC | |
| 2 | | UGCG | |
| LwaCas13 | 52 | GAUUUAGACUACCCCAAAAACGAAGGGGAC | LwaCas13 system |
| guide RNA | | UAAAACCGAGCAGCUGAAACUGCGGUGGGA | |
| 3 | | GUCU | |
| LwaCas13 | 53 | GAUUUAGACUACCCCAAAAACGAAGGGGAC | LwaCas13 system |
| guide RNA | | UAAAACCUUGACCAAAUUGGGAUAAGACUC | |
| 4 | | CCAC | |
| LbuCas13 | 54 | GACCACCCCAAAAAUGAAGGGGACUAAAAC | LbuCas13 system |
| guide RNA | | AAGGACAUUCAAAGCCAAUUCGAGCAGCU | |
| 1 | | | |
| LbuCas13 | 55 | GACCACCCCAAAAAUGAAGGGGACUAAAAC | LbuCas13 system |
| guide RNA | | AAAAGCCAAUUCGAGCAGCUGAAACUGCG | |
| 2 | | | |
| LbuCas 13 | 56 | GACCACCCCAAAAAUGAAGGGGACUAAAAC | LbuCas13 system |
| guide RNA | | ACGAGCAGCUGAAACUGCGGUGGGAGUCU | |
| 3 | | | |
| LbuCas13 | 57 | GACCACCCCAAAAAUGAAGGGGACUAAAAC | LbuCas13 system |
| guide RNA | | ACUUGACCAAAUUGGGAUAAGACUCCCAC | |
| 4 | | | |

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

### NON-LIMITING WORKING EXAMPLES

The following examples are put forth so as to provide those of ordinary skill in the art with a complete disclosure and description of how to make and use this disclosure, and are not intended to limit the scope of what the inventors regard as their disclosure, nor are they intended to represent or imply that the experiments below are all of or the only experiments performed. It will be appreciated by persons skilled in the art that numerous variations and/or modifications may be made to this disclosure as shown in the specific aspects without departing from the spirit or scope of this disclosure as broadly described. For instance, certain master mixes can in principle be combined; certain enzymes can be replaced by others with similar functionality; certain buffers can be modified depending on the enzyme to be implemented; the number of segments can be altered (e.g., duplicate segments could be implemented in certain configurations of the device and/or certain segments could in principle be combined) without altering the spirit of this disclosure. The present aspects are, therefore, to be considered in all respects as illustrative and not restrictive.

### Example 1—Devices and Systems for Detecting a Target Microorganism

Devices having tubes with a plurality of subsections for coupling nucleic acid amplification tests and CRISPR systems were manufactured as described below:

A series of master mixes comprising reagents for detecting FluA were prepared. For this particular implementation of the device, a first PCR Master Mix was prepared as follows:

TABLE 2
PCR Master Mix Part 1

| Materials | Stock Concentration | | Concentration in PCR | |
|---|---|---|---|---|
| PCR Water | NA | NA | NA | NA |
| Tricine pH 8.2 | 1000 | mM | 25.89 | mM |
| Potassium Acetate | 2500 | mM | 40.89 | mM |
| dATP | 100 | mM | 0.47 | mM |
| dCTP | 100 | mM | 0.47 | mM |
| dGTP | 100 | mM | 0.47 | mM |
| dUTP | 100 | mM | 0.47 | mM |
| 50% Glycerol | 50 | % (v/v) | 1.30 | % (v/v) |
| rHSA | 100 | mg/mL | 0.33 | mg/mL |
| FS-A-F1 | 1000 | μM | 2.53 | μM |
| FS-A-F2 | 1000 | μM | 0.63 | μM |
| FS-A-F3 | 1000 | μM | 0.63 | μM |
| Tween-20 | 10 | % (v/v) | 0.01 | % (v/v) |
| EDTA | 25 | mM | 0.02 | mM |
| Sodium Azide | 10 | % (w/v) | 0.01 | % (w/v) |
| DMSO | 100 | % (v/v) | 0.37 | % (v/v) |
| Tris-HCl pH 7.0 | 100 | mM | 3.29 | mM |
| DTT | 100 | mM | 0.12 | mM |
| Tween-80 | 1 | % | 0.00 | % |
| NS MMLV | 85 | U/μL | 0.54 | U/μL |
| EDTA | 500 | mM | 0.35 | mM |
| UNG | 50 | U/μL | 0.17 | U/μL |

For this particular implementation of the device, a second PCR Master Mix was prepared as follows:

TABLE 3
PCR Master Mix Part 2

| Materials | Stock Concentration | | Concentration in PCR | |
|---|---|---|---|---|
| PCR Water | NA | NA | NA | NA |
| Tricine pH 8.2 | 1000 | mM | 25.89 | mM |
| Potassium Acetate | 2500 | mM | 40.89 | mM |
| rHSA | 100 | mg/mL | 0.33 | mg/mL |
| FS-A-R1 | 1000 | μM | 1.26 | μM |
| FS-A-P1 | 200 | μM | 0.16 | μM |
| Tween-20 | 10 | % (v/v) | 0.01 | % (v/v) |
| EDTA | 25 | mM | 0.02 | mM |
| Sodium Azide | 10 | % (w/v) | 0.01 | % (w/v) |
| One Step Aptamer | 200 | μM | 3.89 | μM |
| Enzyme ZO5 SP PZ | 200 | U/μL | 4.25 | U/μL |

For various implementations of the device, a plurality of mixes comprising CRISPR enzymes were prepared. In the particular implementation described herein, a CRISPR Master Mix was prepared as follows:

TABLE 4
CRISPR Enzyme Master Mix 1

| | | |
|---|---|---|
| Materials | Stock Concentration | Concentration in PCR |

| | | | | |
|---|---|---|---|---|
| PCR water | NA | NA | NA | NA |
| DnaseAlert | 2000 | nM | 333.33 | nM |
| Lba Cas12 | 10000 | nM | 100.00 | nM |
| Guide RNA | 10000 | nM | 150.00 | nM |
| MgCl₂ | 1000 | mM | 4.00 | mM |
| Tween 20 | 0.1 | v/v | 0.00 | v/v |

In a Quality Control hood, internal controls were prepared as follows:

TABLE 5

| | Stock | Added | Final Conc. |
|---|---|---|---|
| Target Stock | Conc. (xLoD) | Stock (μL) | (xLoD) |
| 1000xFluA | 1000 | 23.40 | 9 |

The remainder reagents were prepared as described below:

TABLE 6

| | | |
|---|---|---|
| Material | Material Number | μL/tube |

Beads

| | | |
|---|---|---|
| LMP | 08485461102 | 12.5 |

Lysis Buffer (abbreviated LB in certain legends)

| | | |
|---|---|---|
| Generic Lysis | 09152431001 | 215 |

Wash Buffer (abbreviated WB in certain legends)

| | | |
|---|---|---|
| Omni Wash Buffer | 06997503190 | 240 |

Elution Buffer (abbreviated EB in certain legends)

| | | |
|---|---|---|
| FRTA EB | 07568649001 | 50 |

Each assay tube contains all reagents for a single test, packaged in sequential segments. FIG. 1A is a schematic illustrating one such device prepared with NAAT reagents and CRISPR reagents. FIG. 1B is a schematic illustrating one such state of the art device, namely a device prepared with the reagents described herein, but containing reagents for nucleic acid amplification tests (NAAT) only, without any CRISPR enzymes or CRISPR systems. The devices are illustrated side-by-side for comparison purposes.

The assay tube, which was fabricated from a thermoplastic material suitable for use in amplification reactions, was assembled with approximately the following volumes of the reagents:

TABLE 7
Device 1 (FIG. 1A)

| | | Volume |
|---|---|---|
| Segment | Reagent Information | (μL) |
| 9 | None | 0 |
| 8 | Second PCR Master Mix | 15 |
| 7 | First PCR Master Mix | 15 |
| 6 | Elution | 50 |
| 5 | Wash | 240 |
| 4 | Lysis | 215 |
| 3 | Beads | 12.5 |
| 2 | Internal Control | 12.5 (as needed) |
| 1 | Not applicable (segment | |
| | adapted for receiving a sample) | |

TABLE 8
Device 2 (FIG. 1B)

| | | Volume |
|---|---|---|
| Segment | Reagent Information | (μL) |
| 9 | CRISPR Systems | 60 |
| 8 | Second PCR Master Mix | 15 |
| 7 | First PCR Master Mix | 15 |
| 6 | Elution | 50 |
| 5 | Wash | 240 |
| 4 | Lysis | 215 |
| 3 | Beads | 12.5 |
| 2 | Internal Control | 12.5 (as needed) |
| 1 | Not applicable (segment | |
| | adapted for receiving a sample) | |

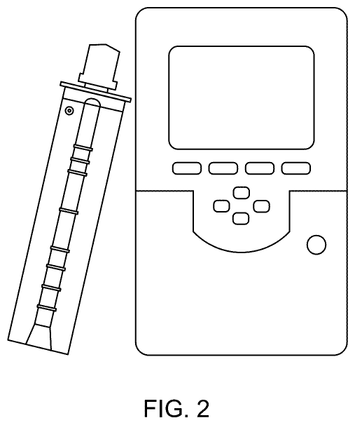

The assay tube, a device, was subsequently mounted on a frame. For this particular embodiment, the frame was an open frame, such as the frame illustrated in FIG. 2 and FIG. 3. FIG. 2 depicts on open frame next to a system for detecting a signal from the assay. On this particular embodiment, the assay tube and the frame were configured for use with Roche Diagnostics® Cobas® omni utility channel, which consolidates in-vitro diagnostics and an open channel assay onto a single platform for detection and reporting of signals detected by the assay. FIG. 3 illustrates a configuration of the device wherein a right side of the frame comprises a grading. In certain systems, such as the Cobas® 6800/8800 system, the grading is adapted for facilitating a sliding of the device into a system that provides a signal readout.

### Example 2—Validation of Devices and Systems for Detecting a Target Microorganism Inclusivity Test

A number of devices were manufactured as described in Example 1. To test the performance of the device in detecting FluA viruses that can comprises certain nucleic acid sequence changes, a plurality of FluA test synthetic ssDNA target sequences with single mismatches (37 positions+WT) were synthesized. They correspond to the sequences listed in SEQ ID NO: 1 and SEQ ID NOs: 9-47. Four replicates of each FluA DNA target sequence with single mismatches (37 positions+WT) were tested follows:

### Negative Control Validation Workflow:

A user pressed the power on/off button to start the Cobas® Liat® Analyzer system.

A user selected “Login” on the screen of the Cobas® Liat® Analyzer system and entered the relevant authentication information.

A user selected “Assay Menu” on the main menu of a Cobas® Liat® Analyzer system and selected “New Lot” at the bottom of the list.

When prompted to Scan the Insert ID, the user selected “Scan” and scanned the negative control package and the Negative Control Barcode card typically included with the control kit. Ensure that the red scan light is over the entire barcode. Next, the Cobas® Liat® analyzer system prompted the message “Add negative control & scan tube ID.”

The user held a tube of Negative Control upright and lightly tapped on a flat surface to collect liquid at the bottom of the tube. The user visually checked that the dilution had pooled at the bottom of the tube.

The user carefully removed the cap of the device to be tested and inserted the pipette into the opening. The user slowly squeezed the bulb to empty the contents of the negative control sample in the pipette into the device assay tube. The user was careful to avoid creating bubbles in the sample.

The user screwed the cap back onto the assay device tube and disposed of the transfer pipette as biohazardous material. The user then selected “scan” and place the assay tube horizontally on the table beneath a barcode reader so that a red scan light was over the entire barcode. The tube entry door on top of the system (i.e., analyzer system) automatically opened once the barcode was read.

The user removed the assay tube sleeve and immediately inserted the assay tube into the Cobas® Liat® Analyzer until the tube clicked into place. In the Cobas® Liat® analyzer system the grading or groove of the assay tube only fits on the left while the cap is on top.

Once the assay tube was properly inserted, the Cobas® Liat® analyzer system closed its door automatically and begun the test.

During the test, the Cobas® Liat® Analyzer displayed the running status and estimated time remaining. Once the test was completed, the Cobas® Liat® analyzer system displayed the message, “Remove tube slowly and carefully” and opened the tube entry door automatically. A message displaying “negative control result accepted” was displayed at the end of the run. The user confirmed.

### Positive Control Validation Workflow:

A plurality of positive controls with various FluA test synthetic ssDNA target sequences with single mismatches (37 positions+WT) (see, e.g., SEQ ID NOs: 9-47 and SEQ ID NO: 1) were prepared for analysis by the devices and systems of the disclosure.

Briefly, after the synthetic sequences in the positive control tube(s) were allowed to re-dissolve, steps analogous to the ones performed for the negative control tube were undertaken by a user. The positive control samples were then run as tests.

In this particular implementation detection of FluA as the test microorganism was tested from devices for dual detection of FluA via NAAT and CRISPR detection. This particular experiment evaluated the performance of a device described in Example 1 for detection of FluA wild-type and single mismatch sequences.

### Results:

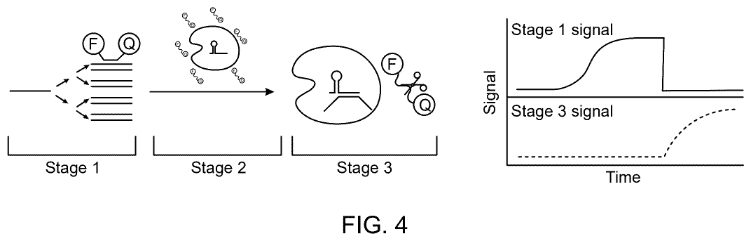

FIG. 4 is a schematic illustrating three distinct stages in which signals are produced from a device of the disclosure and detected by a systems analyzer in one integrated process. As shown in FIG. 4, the fluorescent signal for the NAAT stage and the CRISPR stage of the process is detected at distinct points in time. Stage 1 illustrates the NAAT process with NAAT signal readout. The fluorescent signal from NAAT is detected before a reaction from the CRISPR enzyme is performed. Stage 2 illustrates the liquid handling process to enable amplicon transfer and interaction with CRISPR systems. Stage 3 illustrates the CRISPR process with CRISPR signal readout, which comes after the stage 1 signal is produced.

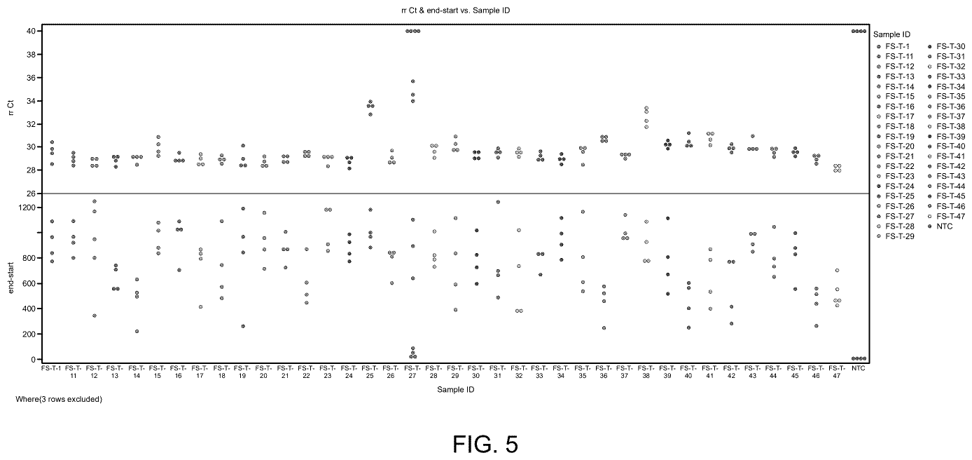

FIG. 5 provides the results of the inclusivity experiments evaluating the ability of the system and methods to inclusively detect FluA test synthetic ssDNA target sequences with single mismatches (37 positions+WT) (see, e.g., SEQ ID NOs: 9-47 and SEQ ID NO: 1). As shown in FIG. 5, good inclusively was generally observed for both CRISPR systems and NAAT modes of detection (demonstrated by PCR Taqman probe), thus validating the devices and systems described herein for detection of FluA in wild type sequences and with a number of single mismatched sequences.

### Example 3—Validation of Devices and Systems for Detecting a Target Microorganism Sensitivity Test

The sensitivity of a NAAT refers to its ability to amplify small amounts of target nucleic acid (DNA or RNA) in a sample. CRISPR sensitivity refers to the ability of the enzyme to accurately and specifically target a particular target sequence.

To probe the sensitivity of the devices described herein, a plurality of devices were manufactured as described in Example 1. A program similar to the program described in Example 2 was executed, except that only a wild type sequence was used for evaluation of sensitivity. For consistency purposes, the devices were tested on ten different Cobas® Liat® analyzer systems. Briefly, a generalized linear model fit was used to fit a known concentration of FluA synthetic WT sequence (SEQ ID NO: 1) as detected by the CRISPR channel(s) and the NAAT channel(s).

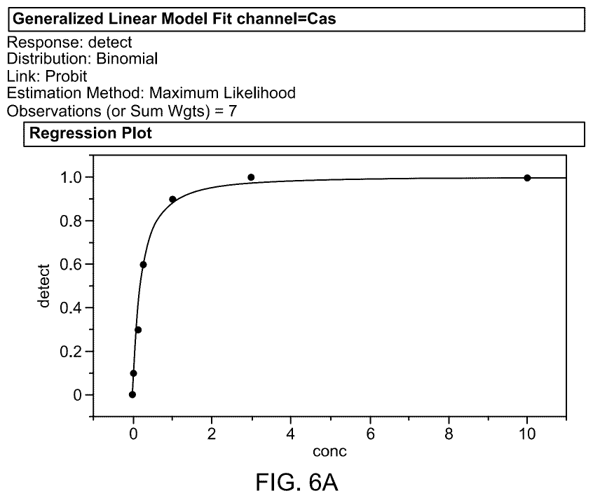

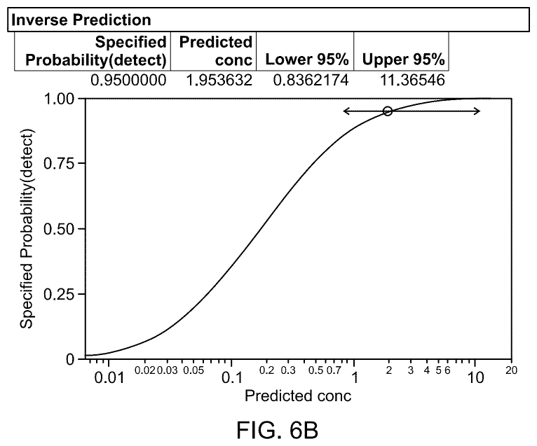

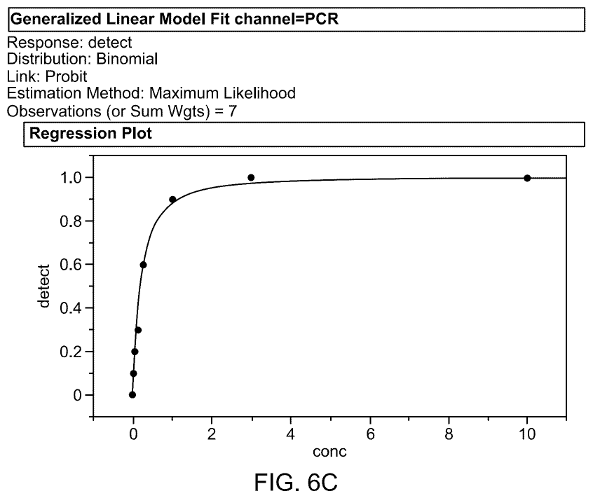

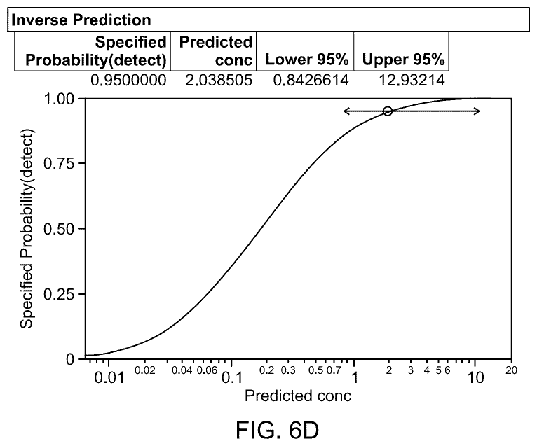

The results of the test are listed in FIGS. 6A-6D. The figures are charts illustrating: (A) generalized linear model fits detected in the CRISPR channel (Cas), along with its inverse prediction (B); and (C) generalized linear model fits detected in the CRISPR channel (Cas), along with its inverse prediction (D). The summary of the sensitivity evaluation is reproduced below:

TABLE 9

| | Specified | | | |
|---|---|---|---|---|
| | Probability | Predicted | Lower | Upper |
| Channel | (detect) | Concentration | 95% | 95% |
| CRISPR | 0.9500000 | 1.953632 | 0.8362174 | 11.36546 |
| (Cas12) | | | | |
| NAAT | 0.9500000 | 2.038505 | 0.8426614 | 12.93214 |
| (PCR) | | | | |

The data indicate that both NAAT and CRISPR modes of detection performed with a similar sensitivity.

### Example 4—Validation of Devices and Systems for Detecting a Target Microorganism Cas Variation Test

To experimentally evaluate CRISPR variation reactions the following master mixes were prepared and manufactured into devices:

TABLE 10

| | | | | |
|---|---|---|---|---|
| | Material | stock | final | |
| Number | Description | conc | conc | unit |

CRISPR Enzyme Master Mix 1

| | | | | |
|---|---|---|---|---|
| BCCH4252 | PCR water | | | |
| 000791443 mix | DnaseAlert | 2.00E+03 | 500.00 | nM |
| 10156778 | Lba Cas12 (NEB), | 10000 | 150.00 | nM |
| | (10x dilution) | | | |
| 522239254 | Guide RNA | 1.00E+04 | 225.00 | nM |
| | (FS-guide 1) | | | |
| BCCD1631 | MgCl2 | 1.00E+03 | 6.00 | mM |
| 20518A | Tween 20 | 10% | 0.05% | v/v |

| | | | | | |
|---|---|---|---|---|---|
| | Material | stock | final | | volume per |
| Lot # | Description | conc | conc | unit | reaction |

CRISPR Enzyme Master Mix V2.2

| | | | | | |
|---|---|---|---|---|---|
| 000791443 mix | DnaseAlert | 2.00E+03 | 125 | nM | 3.750 |
| 10156778 | Lba Cas12a | 1.00E+04 | 40 | nM | 0.240 |
| | (NEB), M0653T | | | | |
| 522239254 | guide RNA | 1.00E+03 | 22.5 | nM | 1.350 |
| | (FS-guide1) | | | | |
| BCCD1631 | MgCl2 | 1.00E+03 | 9 | mM | 0.540 |
| BCCH4252 | Water | | | | 54.120 |
| | Total | | | | 60.000 |

CRISPR Enzyme Master Mix V2.3

| | | | | | |
|---|---|---|---|---|---|
| 000791443 mix | DnaseAlert | 2.00E+03 | 500 | nM | 15.000 |
| 10156778 | Lba Cas12a | 1.00E+04 | 150 | nM | 0.900 |
| | (NEB), M0653T | | | | |
| 522239254 | guide RNA | 1.00E+04 | 225 | nM | 1.350 |
| | (FS-guide1) | | | | |
| 20518A | Tween 20 | 10% | 0.05% | v/v | 0.300 |
| BCCH4252 | Water | | | | 42.450 |
| | Total | | | | 60.000 |

For this particular implementation of the device, a first core PCR Master Mix was prepared as follows:

TABLE 11

| | | First Core Master Mix 1 (Segments 7 or 8 or another suitable segment) | | | | | | | |
|---|---|---|---|---|---|---|---|---|---|
| Material | Lot | Material | Stock | | Conc in | | Conc in | | μL/ |
| Number | Number | Description | Concentration | | MMX | | PCR | | tube |
| DoA; Aug. 26, 2022BK | 20209A | Tricine pH 8.2 | 1000 | mM | 164.00 | mM | 25.89 | mM | 2.460 |
| DoA; May 11, 2022BK | 10722A | Potassium | 2500 | mM | 259.00 | mM | 40.89 | mM | 1.534 |
| | | Acetate | | | | | | | |
| 1889516102 | 437648000 | dATP | 100 | mM | 3.00 | mM | 0.47 | mM | 0.450 |
| 1889508102 | 46813900 | dCTP | 100 | mM | 3.00 | mM | 0.47 | mM | 0.450 |
| 1889524102 | 40325200 | dGTP | 100 | mM | 3.00 | mM | 0.47 | mM | 0.450 |
| 1889532102 | 49209500 | dUTP | 100 | mM | 3.00 | mM | 0.47 | mM | 0.450 |
| from SHBL6831 | DOM Sep. 26, 2022 BK | 50% Glycerol | 50 | % (v/v) | 8.21 | % (v/v) | 1.30 | % (v/v) | 2.464 |
| 07522690001 | G00580 | rHSA | 100 | mg/mL | 2.07 | mg/mL | 0.33 | mg/mL | 0.311 |
| 20518A | DOM May 19, 2022 BK | Tween-20 | 10 | % (v/v) | 0.06 | % (v/v) | 0.01 | % (v/v) | 0.090 |
| 19 Oct. 2018 | 81019A | EDTA | 25 | mM | 0.15 | mM | 0.02 | mM | 0.090 |
| S020901F2101 | DOA Apr. 27, 2022 | Sodium Azide | 10 | % (w/v) | 0.09 | % (w/v) | 0.01 | % (w/v) | 0.135 |
| 5163129001 | X03752, DOAJun. 28, 2022 | DMSO | 100 | % (v/v) | 2.35 | % (v/v) | 0.37 | % (v/v) | 0.352 |

To this first core master mix the reagents below were added, thereby forming a first master mix.

TABLE 12

| | | First Master Mix (Segments 7 or 8 or another suitable segment) | | | | | | | |
|---|---|---|---|---|---|---|---|---|---|
| Material | Lot | Material | Stock | | Conc in | | Conc in | | μL/ |
| Number | Number | Description | Concentration | | MMX | | PCR | | tube |
| | Feb. 16, 2023 | First Core Master | | mM | | mM | | mM | 9.255 |
| | | Mix I | | | | | | | |
| 426406051 | 400747498 | FS-A-F1 | 1000 | μM | 16.00 | μM | 2.53 | μM | 0.240 |
| 426406052 | 400747499 | FS-A-F2 | 1000 | μM | 4.00 | μM | 0.63 | μM | 0.060 |
| 426406053 | 400747500 | FS-A-F3 | 1000 | μM | 4.00 | μM | 0.63 | μM | 0.060 |
| 8919046001 | 4350PAKTA5 | FLUB-20170718-F1 | 1000 | μM | 8.00 | μM | 1.26 | μM | |
| | | PCR water | | | | | | | 5.385 |

For this particular implementation of the device, a second PCR Master Mix was prepared as follows:

TABLE 13

| | | Second Master Mix | | | | | | | |
|---|---|---|---|---|---|---|---|---|---|
| | | (Segment 8 or 9 or another suitable segment) | | | | | | | |
| Material | Lot | Material | Stock | | Conc in | | Conc in | | μL/ |
| Number | Number | Description | Concentration | | MMX | | PCR | | tube |
| W4502-1L | RNBK3898 | PCR Water | | | | | | | 10.749 |
| from | DOM: | Tris pH 7 | 100 | mM | 18.667 | mM | 2.95 | mM | 2.800 |
| T107015L20011901 | Sep. 8, 2021 | | | | | | | | |
| from SLCJ6975 | DOM: | DTT | 100 | mM | 0.67 | mM | 0.11 | mM | 0.100 |
| | 10914BK | | | | | | | | |
| from BCCD6595 | DOM: | Tween-80 | 1 | % | 0.01 | % | 0.00 | % | 0.100 |
| | Nov. 5, 2022 | | | | | | | | |
| 07144563001 | 63158600, | NS MMLV | 85 | U/μL | 3.06 | U/μL | 0.48 | U/μL | 0.540 |
| | DOA: | | | | | | | | |
| | Aug. 18, 2022BK | | | | | | | | |
| 15575-038 | 2393343 | EDTA | 500 | mM | 2.00 | mM | 0.32 | mM | 0.060 |
| 20-02952 | 00729A by JC | ZO5 storage | | | | | | | 0.360 |
| | | buffer | | | | | | | |
| 07006187001 | H03723 | UNG | 50 | U/μL | 0.97 | U/μL | 0.15 | U/μL | 0.291 |

For this particular implementation of the device, a third core PCR Master Mix was prepared as follows:

TABLE 14

| | | Third Core Master Mix 3 (last segment) | | | | | | | |
|---|---|---|---|---|---|---|---|---|---|
| Material | Lot | Material | Stock | | Conc in | | Conc in | | μL/ |
| Number | Number | Description | Concentration | | MMX | | PCR | | tube |
| DoA; May 11, 2022BK | 10722A | Potassium Acetate | 2500 | mM | 259.00 | mM | 25.89 | mM | 1.554 |
| DoA; Aug. 26, 22BK | 20209A | Tricine pH 8.2 | 1000 | mM | 164.00 | mM | 40.89 | mM | 2.460 |
| 07522690001 | G00580 | rHSA | 100 | mg/mL | 2.07 | mg/mL | 0.33 | mg/mL | 0.311 |
| 20518A | DOM May 19, 2022 BK | Tween-20 | 10 | % (v/v) | 0.06 | % (v/v) | 0.01 | % (v/v) | 0.090 |
| 19 Oct. 2018 | 81019A | EDTA | 25 | mM | 0.15 | mM | 0.02 | mM | 0.090 |
| S020901F2101 | DOA Apr. 27, 2022 | Sodium Azide | 10 | % (w/v) | 0.09 | % (w/v) | 0.01 | % (w/v) | 0.135 |
| 09384243001 | H06738 | One Step Aptamer | 200 | μM | 24.62 | μM | 3.89 | μM | 1.846 |
| 7354720001 | H03725, DOAAug. 5, | Enzyme ZO5 SP PZ | 200 | U/μL | 26.93 | U/μL | 4.25 | U/μL | 2.020 |
| | 2022BK | | | | | | | | |

To this third core master mix the reagents below were added, thereby forming a third master mix.

TABLE 15

| | | Third Master Mix (Segment 9 or 10; or another suitable segment) | | | | | | | |
|---|---|---|---|---|---|---|---|---|---|
| Material | Lot | Material | Stock | | Conc in | | Conc in | | μL/ |
| Number | Number | Description | Concentration | | MMX | | PCR | | tube |
| | Feb. 16, 2023 | Seg 10 core | | mM | | mM | | mM | 8.505 |
| 426406054 | 400747501 | FS-A-R1 | 1000 | μM | 8.00 | μM | 1.26 | μM | 0.120 |
| 8913030001 | 4334AKTA5P | FLUB-20170718-R1 | 1000 | μM | 8.00 | μM | 1.26 | μM | |
| 426406055 | 522198120 | FS-A-P1 | 200 | μM | 1.00 | μM | 0.16 | μM | 0.075 |
| 9624139001 | 4417AKTA5P | FluB-20170718-P1_Q12 | 200 | μM | 2.00 | μM | 0.32 | μM | |
| | | PCR water | | | | | | | 6.300 |

In a Quality Control hood, quality control reagents were prepared as follows:

TABLE 16

| 1000x FluA target stock | μL | μL |
|---|---|---|
| 1000x | 1 | 5 |
| UTM | 99 | 495 |

The remainder reagents were prepared as described below:

TABLE 17

| | | |
|---|---|---|
| Material | Material Number | μL/tube |

Beads

| | | |
|---|---|---|
| LMP | 08485461102 | 12.5 |

Lysis Buffer (abbreviated LB in certain legends)

| | | |
|---|---|---|
| Generic Lysis | 09152431001 | 215 |

Wash Buffer (abbreviated WB in certain legends)

| | | |
|---|---|---|
| Omni Wash Buffer | 06997503190 | 240 |

Elution Buffer (abbreviated EB in certain legends)

| | | |
|---|---|---|
| FRTA EB | 07568649001 | 50 |

Each assay tube contains all reagents for a single test, packaged in sequential segments, as previously illustrated in FIG. 1A.

The assay tube, which was fabricated from a thermoplastic material suitable for use in amplification reactions, was assembled with approximately the following volumes of the reagents:

TABLE 18
Device

| | | Volume |
|---|---|---|
| Segment | Reagent Information | (μL) |
| 10 | Master Mix 3 | 60 |
| 9 | Master Mix 2 | 15 |
| 8 | Master Mix 1 | 30 |
| 7 | Elution | 50 |
| 5-6 | Wash | 240 |
| 3-4 | Lysis | 215 |
| 2 | Beads | 12.5 |
| 1 | Not applicable (segment | |
| | adapted for receiving a sample) | |

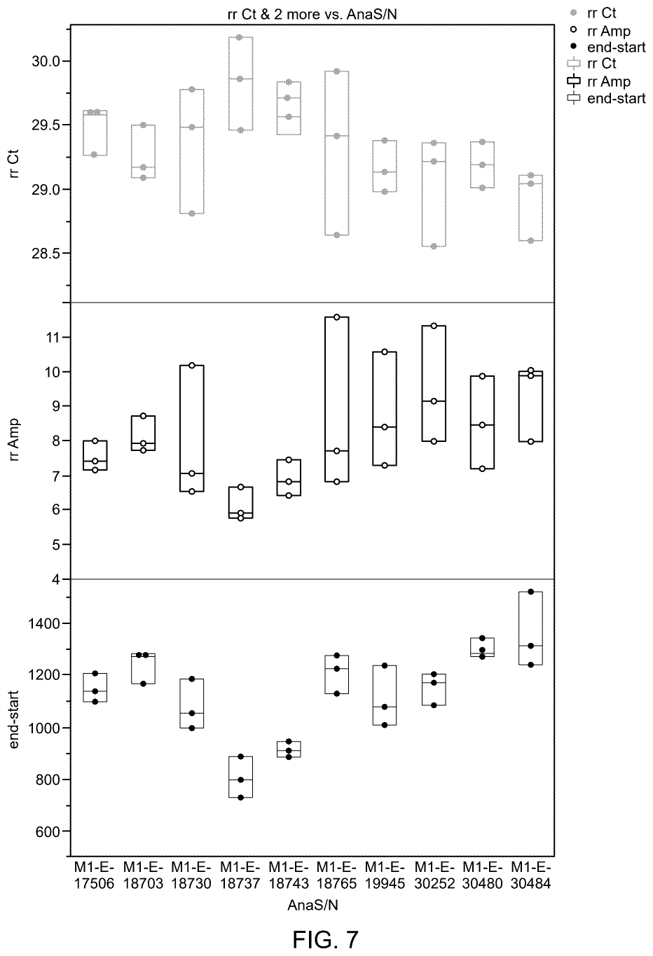

The devices were tested on 10 different analyzer systems to evaluate any potential intra-device inconsistency. As illustrated in FIG. 7, the CRISPR variation observed on 10 distinct analyzers provides an acceptable range for NTC and FluA. The average EndPoint-Start CRISPR gg of cas on 10×Flu A is 1130, cv is 15.6% in 10 analyzers. The average Ave EndPoint-Start CRISPR gg of cas NTC is 2.9 from 10 analyzers. The results displayed in FIG. 7 were obtained with a device manufactured with CRISPR Enzyme Master Mix 1 on its last segment.

### Example 5—Validation of Devices and Systems for Detecting a Plurality of Target Microorganism

Having validated a dual-detection system (NAAT+CRISPR) for detection of FluA, the disclosure considered validation of devices having assay tubes with a plurality of subsections for coupling nucleic acid amplification tests and CRISPR systems for the detection of a plurality of target microorganism.

Rapid and accurate diagnosis and differentiation of SARS-CoV-2, RSV, and influenza (type A and type b) infections is important in individuals suspected of a respiratory infection. The seasonality of COVID-19 and influenza overlap and the clinical manifestations of these diseases can be similar, ranging from asymptomatic or mild “influenza-like” illness (such as fever, cough, shortness of breath, or myalgia) in a majority of individuals to more severe and life-threatening disease. The current widespread implementation of rapid point of care (POC) testing for influenza underscores the importance of prompt and accurate.

Non-optimal oligo design or challenging reaction conditions could lead to false positive (specificity concern) or false negative (inclusivity concern) signal in either NAAT or CRISPR and cause misidentification of target pathogens. Due to the difference in the target recognition mechanism of the two systems, the possibility of an integrated device and method applying both NAAT and CRISPR detection systems detecting simultaneous faulty signals is low when targeting the same pathogens compared to single systems.

Therefore, the NAAT+CRISPR dual-detection device, systems, and methodology developed and described in the instant case system can improve the specificity and inclusivity of the assay.

A series of master mixes comprising reagents for detecting FluA, FluB, RSV-A, SARS-CoV2 were prepared. For this particular implementation of the device targeting FluA, a first PCR Master Mix is prepared as follows:

TABLE 19
Master Mix 1

| Material | Stock | | Conc in | | Conc in | | μL/ | Amount of rxn | |
|---|---|---|---|---|---|---|---|---|---|
| Description | Concentration | | MMX | | PCR | | tube | 50 | |
| PCR Water | | | | | | | 5.022 | 251.1 | uL |
| Tricine pH 8.2 | 1000 | mM | 164.00 | mM | 25.89 | mM | 2.460 | 123.0 | uL |
| Potassium Acetate | 2500 | mM | 259.00 | mM | 40.89 | mM | 1.554 | 77.7 | uL |
| dATP | 100 | mM | 3.00 | mM | 0.47 | mM | 0.450 | 22.5 | uL |
| dCTP | 100 | mM | 3.00 | mM | 0.47 | mM | 0.450 | 22.5 | uL |
| dGTP | 100 | mM | 3.00 | mM | 0.47 | mM | 0.450 | 22.5 | uL |
| dUTP | 100 | mM | 3.00 | mM | 0.47 | mM | 0.450 | 22.5 | uL |
| 50% Glycerol | 50 | % (v/v) | 8.21 | % (v/v) | 1.30 | % (v/v) | 2.464 | 123.2 | uL |
| rHSA | 100 | mg/mL | 2.07 | mg/mL | 0.33 | mg/mL | 0.311 | 15.5 | uL |
| A_RT3 | 399 | μM | 16.00 | μM | 2.53 | μM | 0.602 | 30.1 | uL |
| A_RT4 | 993 | μM | 4.00 | μM | 0.63 | μM | 0.060 | 3.0 | uL |
| A_RT5 | 997 | μM | 4.00 | μM | 0.63 | μM | 0.060 | 3.0 | uL |
| Tween-20 | 10 | % (v/v) | 0.06 | % (v/v) | 0.01 | % (v/v) | 0.090 | 4.5 | uL |
| EDTA | 25 | mM | 0.15 | mM | 0.02 | mM | 0.090 | 4.5 | uL |
| Sodium Azide | 10 | % (w/v) | 0.09 | % (w/v) | 0.01 | % (w/v) | 0.135 | 6.8 | uL |
| DMSO | 100 | % (v/v) | 2.35 | % (v/v) | 0.37 | % (v/v) | 0.352 | 17.6 | uL |
| | | | | | | | 15.00 | 750.0 | 300 uL |

For this particular implementation of the device targeting FluA, a second PCR Master Mix is prepared as follows:

TABLE 20
Master Mix 2

| Material | Stock | | Conc in | | Conc in | | μL/ | Amount of rxn | |
|---|---|---|---|---|---|---|---|---|---|
| Description | Concentration | | MMX | | PCR | | tube | 50 | |
| PCR Water | | | | | | | 10.749 | 537.5 | uL |
| Tris-HCl pH 7.0 | 100 | mM | 18.67 | mM | 3.29 | mM | 2.800 | 140.0 | uL |
| DTT | 100 | mM | 0.67 | mM | 0.12 | mM | 0.100 | 5.0 | uL |
| Tween-80 | 1 | % | 0.01 | % | 0.00 | % | 0.100 | 5.0 | uL |
| NS MMLV | 85 | U/μL | 3.06 | U/μL | 0.54 | U/μL | 0.540 | 27.0 | uL |
| EDTA | 500 | mM | 2.00 | mM | 0.35 | mM | 0.060 | 3.0 | uL |
| UNG | 50 | U/μL | 0.97 | U/μL | 0.17 | U/μL | 0.291 | 14.6 | uL |
| ZO5 storage buffer | | | | | | | 0.360 | 18.0 | uL |
| | | | | | | | 15.00 | 750.0 | 300 uL |

For this particular implementation of the device targeting FluA, a third PCR Master Mix is prepared as follows:

TABLE 21
Master Mix 3

| Material | Stock | | Conc in | | Conc in | | μL/ | Amount of rxn | |
|---|---|---|---|---|---|---|---|---|---|
| Description | Concentration | | MMX | | PCR | | tube | 50 | |
| PCR Water | | | | | | | 6.299 | 314.9 | uL |
| Tricine pH 8.2 | 1000 | mM | 164.00 | mM | 25.89 | mM | 2.460 | 123.0 | uL |
| Potasskam Acetate | 2500 | mM | 259.00 | mM | 40.89 | mM | 1.554 | 77.7 | uL |
| rHSA | 100 | mg/mL | 2.07 | mg/mL | 0.33 | mg/mL | 0.311 | 15.5 | uL |
| FLUA-M1-R1 | 1002 | μM | 8.00 | μM | 1.26 | μM | 0.120 | 6.0 | uL |
| FLUA_M1_P3E\|213_194\|_Q10.OK | 200 | μM | 1.00 | μM | 0.16 | μM | 0.075 | 3.8 | uL |
| Tween-20 | 10 | % (v/v) | 0.06 | % (v/v) | 0.01 | % (v/v) | 0.090 | 4.5 | uL |
| EDTA | 25 | mM | 0.15 | mM | 0.02 | mM | 0.090 | 4.5 | uL |
| Sodium Azide | 10 | % (w/v) | 0.09 | % (w/v) | 0.01 | % (w/v) | 0.135 | 6.8 | uL |
| One Step Aptamer | 200 | μM | 24.62 | μM | 3.89 | μM | 1.846 | 92.3 | uL |
| Enzyme ZO5 SP PZ | 200 | U/μL | 26.93 | U/μL | 4.25 | U/μL | 2.020 | 101.0 | uL |
| | | | | | | | 15.00 | 750.0 | 300 uL |

CRISPR master mix can include Cas enzyme/guide RNA systems targeting multiple pathogens in a multiplexed setup. In this particular implementation, a CRISPR Master Mix targeting FluA can be prepared as follows:

TABLE 22
CRISPR Enzyme Master Mix 1

| Materials | Stock concentration | | Concentration in PCR | |
|---|---|---|---|---|
| PCR water | NA | NA | NA | NA |
| DnaseAlert | 2000 | nM | 333.33 | nM |
| Lba Cas12 | 10000 | nM | 100.00 | nM |
| Guide RNA | 10000 | nM | 150.00 | nM |
| (targeting FluA) | | | | |
| MgCl₂ | 1000 | mM | 4.00 | mM |
| Tween 20 | 0.1 | v/v | 0.00 | v/v |

Internal control can be added into tube segment 2 as needed. In this particular implementation, no internal control was used.

The remainder reagents were prepared as described below:

TABLE 23

| | | |
|---|---|---|
| Material | Material Number | μL/tube |

Beads

| | | |
|---|---|---|
| LMP | 08485461102 | 12.5 |

Lysis Buffer (abbreviated LB in certain legends)

| | | |
|---|---|---|
| Generic Lysis | 09152431001 | 215 |

Wash Buffer (abbreviated WB in certain legends)

| | | |
|---|---|---|
| Omni Wash Buffer | 06997503190 | 240 |

Elution Buffer (abbreviated EB in certain legends)

| | | |
|---|---|---|
| FRTA EB | 07568649001 | 50 |

Each assay tube contains all reagents for a single test, packaged in sequential segments.

TABLE 24
Device

| Segment | Reagent Information |
|---|---|
| 9 | CRISPR Systems (FluA) |
| 8 | Third PCR Master Mix |
| 7 | First and Second PCR Master Mix |
| 6 | Elution |
| 5 | Wash |
| 4 | Lysis |
| 3 | Beads |
| 2 | Internal Control |
| 1 | Not applicable (segment adapted for receiving a sample) |

The present technology is not to be limited in terms of the particular implementations described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular implementations only, and is not intended to be limiting.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A device, comprising:
an assay tube subdivided into a plurality of subsections, whereby:
a first subsection is connected to an openable cap, whereby an opening accessible from the openable cap in the first subsection is adapted for receiving a sample, whereby the first subsection is sealed from a subsequent subsection along the assay tube;
a second subsection comprising a solution with an internal control, whereby the second subsection is sealed from a subsequent subsection along the assay tube;
a third subsection comprising a solution with magnetic beads in solution, whereby the third subsection is sealed from a subsequent subsection along the assay tube;
a fourth subsection comprising a solution with a lysis buffer, whereby the fourth subsection is sealed from a subsequent subsection along the assay tube;
a fifth subsection comprising a solution with a wash buffer, whereby the fifth subsection is sealed from a subsequent subsection along the assay tube;
a sixth subsection comprising a solution with an elution buffer, whereby the sixth subsection is sealed from a subsequent subsection along the assay tube;
a seventh subsection comprising a solution with a first PCR master mix, hereby the seventh subsection is sealed from a subsequent subsection along the assay tube;
an eighth subsection comprising a solution with a second PCR master mix, whereby the eighth subsection is sealed from a subsequent subsection along the assay tube; and
a ninth tube comprising a solution with a CRISPR enzyme, whereby the ninth subsection is sealed from an end of the assay tube.

2. The device of claim 1, wherein the first PCR master mix is a solution comprising a reaction buffer, dNTP, RT-PCR primers, a MMLV RT polymerase, and Uracil-DNA glycosylase.

3. The device of claim 1, wherein the second PCR master mix is a solution comprising a reaction buffer, a Z05 polymerase and its aptamer, and RT-PCR primers.

4. The device of claim 1, wherein the CRISPR enzyme is a solution comprising CRISPR-Cas enzymes with trans-cleavage activity such as Cas12 and/or Cas13, a guide RNA, substrates, and metal ion co-factors.

5. The device of claim 1, wherein the sample preparation reagents extracts nucleic acid materials from influenza A virus, influenza B virus, Respiratory Syncytial Virus (RSV), and SARS-CoV-2 virus.

6. The device of claim 1, wherein the tube further comprises a tenth subsection comprising a solution with a CRISPR enzyme, whereby the tenth subsection is sealed from an end of the tube.

7. The device of claim 6, wherein the CRISPR enzyme is part of a CRISPR system adapted for specifically cleaving a plurality of nucleic acids that are amplified by PCR reaction.

8. A method for dual-detection of a target microorganism comprising:
(a) amplifying a first target nucleic acid sequence in a nucleic acid amplification reaction and detecting a signal from the nucleic acid amplification of the first target nucleic acid sequence;
(b) detecting a second target nucleic acid sequence with a CRISPR system targeting the second target nucleic acid sequence and detecting a signal from the second target nucleic acid activation and substrate cleavage by the CRISPR system;
(c) comparing the signal detected from the nucleic acid amplification of the first target nucleic acid sequence with an amplification threshold and comparing the signal detected from the second target nucleic acid activation and substrate cleavage by the CRISPR system with a CRISPR threshold;
wherein the first target nucleic acid sequence and the second target nucleic acid sequence are sequences from the target microorganism;
whereby the target nucleic acid sequence is dually-detected when the signal detected from the first target nucleic acid amplification reaction and the signal detected from the second target nucleic acid activation and substrate cleavage by the CRISPR system are both above the threshold.

9. The method of claim 8, wherein the nucleic acid amplification reaction is selected from the group of reactions consisting of: a polymerase chain reaction (PCR), a loop mediated isothermal amplification (LAMP) reaction, a recombinase polymerase amplification (RPA) reaction, a nucleic acid sequence based amplification (NASBS) reaction, a transcription mediated amplification (TMA) reaction, a rolling circle amplification (RCA) reaction, a strand displacement amplification (SDA) reaction, a nicking and extension amplification reaction (NEAR), an exponential amplification reaction (EXPAR), a multiple displacement amplification (MDA) reaction, a helicase dependent amplification (HAD) reaction, a hybridization chain reaction (HCR).

10. The method of claim 9, wherein the CRISPR system is a system with trans-cleavage nuclease activity.

11. The method of claim 10, wherein the system with trans-cleavage activity is selected from the group of systems consisting of: a Cas12 system and a Cas13 system.

12. The method of claim 8, wherein the first target nucleic acid sequence is a FluA sequence.

13. The method of claim 8, wherein the second target nucleic acid sequence is FluA sequence.

14. The method of claim 8, wherein a region of the first target nucleic acid amplification sequence comprises the second target nucleic acid sequence.

15. The method of claim 14, wherein the first target nucleic acid sequence comprises a plurality of regions comprising the second target nucleic acid sequence(s).

16. The method of claim 15, wherein activation and substrate cleavage of the plurality of second target nucleic acid sequence(s) further amplifies the signal detected from the second target nucleic acid by a factor that is directly proportional to the number of the plurality of second target nucleic acid sequences.

17. The method of claim 8, wherein the method comprises amplifying a plurality of first target nucleic acid sequences for multiplex detection of a plurality of microorganisms.

18. The method of claim 8, wherein the method further comprises a step of cleaving a plurality of nucleic acids with a non-specific nuclease after detection of the amplification threshold and the CRISPR threshold thereby reducing nucleic acid contamination in the system.

19. A method for detecting a target nucleic acid sequence, comprising:
(a) adding a sample into a device comprising:
an assay tube subdivided into a plurality of subsections, whereby a first subsection is connected to an openable cap, whereby an opening accessible from the openable cap in the first subsection is adapted for receiving a sample, whereby the first subsection is sealed from a subsequent subsection along the tube;
a second subsection comprising a solution with internal control reagents, whereby the second subsection is sealed from a subsequent subsection along the tube;
a third subsection comprising a solution with magnetic beads in solution, whereby the third subsection is sealed from a subsequent subsection along the tube;
a fourth subsection comprising a solution with a lysis buffer, whereby the fourth subsection is sealed from a subsequent subsection along the tube;
a fifth subsection comprising a solution with a wash buffer, whereby the fifth subsection is sealed from a subsequent subsection along the tube;
a sixth subsection comprising a solution with an elution buffer, whereby the sixth subsection is sealed from a subsequent subsection along the tube;
a seventh subsection comprising a solution with a PCR master mix part 1, whereby the seventh subsection is sealed from a subsequent subsection along the tube;
an eighth subsection comprising a solution with a PCR master mix part 2, whereby the eighth subsection is sealed from a subsequent subsection along the tube; and
a ninth tube comprising a solution with a CRISPR enzyme, whereby the ninth subsection is sealed from an end of the tube.
(b) adding the device comprising the sample into a system that supports a sequential rupture of one subsection from a subsequent subsection, thereby allowing the reagents in one subsection to sequentially react with the reagents in the subsequent subsection; and
(c) detecting the target nucleic acid by detecting a signal from the last subsection.
